(19) United States
(12) Patent Application Publication
(10) Pub. No.: US 2012/0192311 A1
(43) Pub. Date: Jul. 26, 2012

(54) RIPENING PROMOTER

(75) Inventors: Geza Hrazdina, Geneva, NY (US); Mehmet Karaaslan, Sanliurfa (TR)

(73) Assignee: Cornell Center for Technology, Enterprise & Commercialization, Ithaca, NY (US)

(21) Appl. No.: 13/359,370

(22) Filed: Jan. 26, 2012

Related U.S. Application Data
(63) Continuation of application No. PCT/US2010/002110, filed on Jul. 28, 2010.
(60) Provisional application No. 61/276,659, filed on Sep. 15, 2009.
(60) Provisional application No. 61/271,883, filed on Jul. 28, 2009.

(51) Int. Cl. A01H 1/06; A01H 5/10; C12N 5/10; A01H 5/00; C12N 15/113; C12N 15/82

(52) U.S. Cl. 800/279; 536/24.1; 435/320.1; 435/419; 800/298; 800/300; 800/301; 800/302; 800/278; 800/289; 435/468

(57) ABSTRACT

The presence of expansins was investigated in various developmental and ripening stages of cherry fruits by SDS PAGE and immunoblotting. An expansin gene and three fragments (242, 607, and 929 bp) of its promoter region were cloned. The genomic clone of the expansin gene contained three introns, two exons spanning a 1.6 kb and a 1.0 kb upstream region. Semi quantitative PCR analysis showed that this gene was ripening specific Chimeric promoter—GUS constructs were made and truncated forms of the expansin promoter were introduced into tomatoes by agroinjection and fruits were analyzed for GUS expression by histochemical GUS staining and enzyme activity assays. The 0.60 kb expansin promoter efficiently induced GUS expression in transgenic tomatoes, whereas constructs with the 0.25 kb promoter did not display significant GUS staining. The highest GUS activity was detected in tomatoes containing the 1.0 kb promoter construct. Both large base pair promoter constructs drove the expression of the GUS gene at an equal or higher rate than the tomato E8 promoter.

### CROSS-REFERENCE TO RELATED APPLICATIONS

This application is a continuation of International Application No. PCT/US2010/002110, filed Jul. 28, 2010, which claims priority from U.S. Provisional Application No. 61/271,883, filed Jul. 28, 2009 and U.S. Provisional Application No. 61/276,659 filed Sep. 15, 2009, each of which is incorporated herein by reference in its entirety.

### BACKGROUND OF THE INVENTION

The invention relates to the field of plant molecular biology, and more particularly to regulation of gene expression in plants.

One of the major events of fruit ripening is the structural modification of the cell wall. Among the numerous enzymes and proteins found in plant cell walls endo-1,4-β-glucanases, xyloglucan endotransglycosylases, and polygalacturonases has drawn attention in recent years because of their elevated expression and activity occurring concomitantly with fruit softening (Fry et al. 1992; Fry 1989; Nishitani and Tominaga 1992). However, the exact role of these enzymes in the softening process is not clear. Studies on transgenic tomato were not able to show a significant correlation between cell wall hydrolase activity and fruit softening (Giovannoni et al. 1989; Brummel and Harpster 2001). Isolation and characterization of expansins and their genes (McQueen-Mason et al. 1992; Scherban et al. 1995) has led to the recognition that expansin accumulation and action is a prerequisite to cell wall hydrolase activity.

Expansins are small, cell wall localized proteins with a molecular weight range of 25-30 kDa (McQueen-Mason et al. 1992; Cosgrove D J 2000). It is thought that expansins may be involved in cell wall modification either by disrupting the hydrogen bonds between cellulose microfibrils or making carbohydrate polymers more accessible to the cell wall localized hydrolytic enzymes (Cosgrove D J 1992). Rose et al. showed the expression of a fruit and ripening specific expansin gene (LeExp1) in tomatoes (Rose et al. 1997). Antisense suppression of this gene resulted in firmer tomato fruits and thus extended shelf life (Brummell et al. 1999). By contrast, expansin-overexpressed transgenic fruits were less firm than controls and softening occurred before commencement of the ripening reactions. Elevated expansin mRNA accumulation has been also detected in non-climacteric fruits such as grape and strawberry (Harrison et al. 2001) and expansin genes were cloned and sequenced from cherries by Gao et al. (2003). Cherries have been traditionally classified as non climacteric fruits and their ripening do not seem to be under the control of ethylene. Therefore, suppression of expansin accumulation may represent a novel strategy to extend their shelf life.

Cauliflower mosaic virus promoter (35S CaMV) is presently widely used for expressing transgenes in plants. 35S CaMV is a constitutive promoter that expresses the transgene in most tissues and times. Because of this, it is not considered to be a viable promoter for up or down regulation of organ and development specific events. Since the patterns of expression of a chimeric gene (or genes) introduced into a plant are controlled using promoters, there is an ongoing interest in the isolation and identification of novel promoters which are capable of controlling expression of a chimeric gene (or genes).

### SUMMARY OF THE INVENTION

Below we describe the expression pattern of expansin proteins in cherries and isolate a fruit and ripening specific promoter to design an efficient and intelligent plant transformation vector to create higher quality fruits.

Compositions and methods for regulating gene expression in a plant are provided. Such compositions include nucleotide sequences for a promoter that initiates transcription in a specific manner.

The sequences described herein find use in the construction of expression vectors for transformation into plants of interest and as molecular markers. The cherry promoter sequences direct expression of operably linked nucleotide sequences in a tissue-preferred manner, for example, (e.g., are fruit and ripening specific). Therefore, the cherry promoter sequences find use in the tissue-preferred expression of an operably linked nucleotide sequence of interest such as a heterologous sequence. The specific methods used to obtain the cherry promoter are described below.

The invention features isolated or substantially purified nucleic acid compositions. An “isolated” or “purified” nucleic acid molecule, or biologically active portion thereof, is substantially free of other cellular material or culture medium when produced by recombinant techniques, or substantially free of chemical precursors or other chemicals when chemically synthesized. An “isolated” nucleic acid is essentially free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 3 kb, 2 kb, 1kb, 0.75kb, 0.5 kb, 0.4kb, 0.2kb, or 0.1 kb of nucleotide sequences that naturally flank the nucleic acid molecule in genomic DNA of the cell from which the nucleic acid is derived.

In general, the cherry promoters described herein drive the endogenous expression of an expansin gene.

The invention accordingly features isolated nucleic acid molecules comprising the promoter nucleotide sequences set forth in SEQ ID NO:1, 2, or 3. The term “promoter” is intended to mean a regulatory region of DNA usually comprising a TATA box capable of directing RNA polymerase Il to initiate RNA synthesis at the appropriate transcription initiation site for a particular coding sequence. A promoter may additionally include other recognition sequences generally positioned upstream or 5′ to the TATA box, referred to as upstream promoter elements, which influence the transcription initiation rate. It is recognized that having identified the nucleotide sequences for the promoter regions disclosed herein, it is within the state of the art to isolate and identify further regulatory elements in the 5′ untranslated region upstream from the particular promoter regions identified herein. Thus, for example, the promoter regions disclosed herein may further include upstream regulatory elements such as those responsible for tissue and temporal expression of the coding sequence, and enhancers. Promoter elements enabling expression in desired tissues, can be identified, isolated, and used with other core promoters to confer tissue-preferred expression. In this aspect of the embodiments, a “core promoter” is intended to mean a promoter without promoter elements.

In one aspect, the invention features an isolated nucleic acid molecule including a nucleotide sequence selected from the group consisting of: a) a nucleotide sequence including the sequence set forth in SEQ ID NO:1 or a complement thereof; and b) a nucleotide sequence including a fragment of the sequence set forth in SEQ ID NO:1, wherein the sequence initiates transcription in a plant cell; and c) a nucleotide sequence including a sequence having at least 90% sequence identity to the sequence set forth in SEQ ID NO:1, SEQ ID NO:2, or SEQ ID NO:3, wherein the sequence initiates transcription in the plant cell.

In another aspect, the invention features a DNA construct including an aforementioned nucleotide sequence operably linked to a heterologous nucleotide sequence of interest. Exemplary heterologous nucleotide sequences of interest may encode a gene product that confers herbicide, salt, cold, drought, pathogen, or insect resistance or the gene product is of agricultural interest. The invention also features a vector including an aforementioned DNA construct.

In another aspect, the invention features a plant cell having stably incorporated into its genome the aforementioned DNA construct or vector. An exemplary plant cell includes a dicot (e.g., a cherry cultivar). The invention further includes a plant having stably incorporated into its genome the aforementioned DNA construct or vector. Such plants are typically either dicots or monocots.

In other aspects, the invention features a transgenic seed which includes an aforementioned DNA construct or vector.

In another aspect, the invention features a method for expressing a nucleotide sequence in a plant, the method including introducing into a plant a DNA construct, the DNA construct including a promoter and operably linked to the promoter a heterologous nucleotide sequence of interest, wherein the promoter includes a nucleotide sequence selected from the group consisting of: a) a nucleotide sequence including the sequence set forth in SEQ ID NO:1 or a complement thereof; and b) a nucleotide sequence including a fragment of the sequence set forth in SEQ ID NO:1, wherein the sequence initiates transcription in a plant cell; and c) a nucleotide sequence including a sequence having at least 90% sequence identity to the sequence set forth in SEQ ID NO:1, wherein the sequence initiates transcription in the plant cell. In one embodiment, the plant is a dicot and wherein the heterologous nucleotide sequence of interest is selectively expressed in a fruit or ovary. In another embodiment, the heterologous nucleotide sequence encodes a gene product that confers herbicide, salt, cold, drought, pathogen, or insect resistance or the gene product is of agricultural interest.

In another aspect, the invention features method for expressing a nucleotide sequence in a plant cell, the method including introducing into a plant cell a DNA construct including a promoter operably linked to a heterologous nucleotide sequence of interest, wherein the promoter includes a nucleotide sequence selected from the group consisting of: a) a nucleotide sequence including the sequence set forth in SEQ ID NO:1 or a complement thereof; b) a nucleotide sequence including a fragment of the sequence set forth in SEQ ID NO:1, wherein the sequence initiates transcription in a plant cell; and c) a nucleotide sequence including a sequence having at least 90% sequence identity to the sequence set forth in SEQ ID NO:1, wherein the sequence initiates transcription in the plant cell. In one embodiment, the plant cell is from a dicot. In another embodiment, the heterologous nucleotide sequence encodes a gene product that confers herbicide, salt, cold, drought, pathogen, or insect resistance or the gene product is of agricultural interest.

In another aspect, the invention features a method for selectively expressing a nucleotide sequence in a fruit or ovary, the method including introducing into a plant cell a DNA construct, and regenerating a transformed plant from the plant cell, the DNA construct including a promoter and a heterologous nucleotide sequence operably linked to the promoter, wherein the promoter includes a nucleotide sequence selected from the group consisting of: a) a nucleotide sequence including the sequence set forth in SEQ ID NO:1 or a complement thereof; b) a nucleotide sequence including a fragment of the sequence set forth in SEQ ID NO:1, wherein the sequence initiates transcription in a plant cell; and c) a nucleotide sequence including a sequence having at least 90% sequence identity to the sequence set forth in SEQ ID NO:1, wherein the sequence initiates transcription in the plant cell. In one embodiment, expression of the heterologous nucleotide sequence alters the phenotype of the fruit or ovary. In another embodiment, the heterologous nucleotide sequence encodes a gene product that confers herbicide, salt, cold, drought, pathogen, or insect resistance or the gene product is of agricultural interest.

In another aspect, the invention features a method of directing expression of a transcribable polynucleotide sequence in a plant cell including operably linking the promoter of any of the aforementioned polynucleotide sequences and transforming a plant cell with the promoter operably linked to the polynucleotide sequence. In this aspect, the invention includes regenerating a plant from the plant cell.

In yet another aspect, the invention features a method for expressing a nucleotide sequence in a plant, the method including introducing into a plant a DNA construct, the DNA construct including a promoter and operably linked to the promoter a heterologous nucleotide sequence of interest, wherein the promoter includes a nucleotide sequence selected from the group consisting of: a) a nucleotide sequence including the sequence set forth in SEQ ID NO:2 or a complement thereof; and b) a nucleotide sequence including a fragment of the sequence set forth in SEQ ID NO:2, wherein the sequence initiates transcription in a plant cell; and c) a nucleotide sequence including a sequence having at least 90% sequence identity to the sequence set forth in SEQ ID NO:2, wherein the sequence initiates transcription in the plant cell. In one embodiment, the plant is a dicot and the heterologous nucleotide sequence of interest is selectively expressed in a fruit or ovary. In another embodiment, the heterologous nucleotide sequence encodes a gene product that confers herbicide, salt, cold, drought, pathogen, or insect resistance or the gene product is of agricultural interest.

In yet another aspect, the invention features a method for expressing a nucleotide sequence in a plant cell, the method including introducing into a plant cell a DNA construct including a promoter operably linked to a heterologous nucleotide sequence of interest, wherein the promoter includes a nucleotide sequence selected from the group consisting of: a) a nucleotide sequence including the sequence set forth in SEQ ID NO:2 or a complement thereof; and b) a nucleotide sequence including a fragment of the sequence set forth in SEQ ID NO:2, wherein the sequence initiates transcription in a plant cell; and c) a nucleotide sequence including a sequence having at least 90% sequence identity to the sequence set forth in SEQ ID NO:2, wherein the sequence initiates transcription in the plant cell. In one embodiment, the plant cell is from a dicot. In another embodiment, the heterologous nucleotide sequence encodes a gene product that confers herbicide, salt, cold, drought, pathogen, or insect resistance or the gene product is of agricultural interest.

In another aspect, the invention features a method for selectively expressing a nucleotide sequence in a fruit or ovary, the method including introducing into a plant cell a DNA construct, and regenerating a transformed plant from the plant cell, the DNA construct including a promoter and a heterologous nucleotide sequence operably linked to the promoter, wherein the promoter includes a nucleotide sequence selected from the group consisting of: a) a nucleotide sequence including the sequence set forth in SEQ ID NO:2 or a complement thereof; and b) a nucleotide sequence including a fragment of the sequence set forth in SEQ ID NO:2, wherein the sequence initiates transcription in a plant cell; and c) a nucleotide sequence including a sequence having at least 90% sequence identity to the sequence set forth in SEQ ID NO:2, wherein the sequence initiates transcription in the plant cell. In one embodiment, expression of the heterologous nucleotide sequence alters the phenotype of the fruit or ovary. In another embodiment, the heterologous nucleotide sequence encodes a gene product that confers herbicide, salt, cold, drought, pathogen, or insect resistance or the gene product is of agricultural interest.

In another aspect, the invention features a method for expressing a nucleotide sequence in a plant, the method including introducing into a plant a DNA construct, the DNA construct including a promoter and operably linked to the promoter a heterologous nucleotide sequence of interest, wherein the promoter includes a nucleotide sequence selected from the group consisting of: a) a nucleotide sequence including the sequence set forth in SEQ ID NO:3 or a complement thereof; and b) a nucleotide sequence including a fragment of the sequence set forth in SEQ ID NO:3, wherein the sequence initiates transcription in a plant cell; and c) a nucleotide sequence including a sequence having at least 90% sequence identity to the sequence set forth in SEQ ID NO:3, wherein the sequence initiates transcription in the plant cell. In one embodiment, the plant is a dicot and wherein the heterologous nucleotide sequence of interest is selectively expressed in a fruit or ovary. In another embodiment, the heterologous nucleotide sequence encodes a gene product that confers herbicide, salt, cold, drought, pathogen, or insect resistance or the gene product is of agricultural interest.

In another aspect, the invention features a method for expressing a nucleotide sequence in a plant cell, the method including introducing into a plant cell a DNA construct including a promoter operably linked to a heterologous nucleotide sequence of interest, wherein the promoter includes a nucleotide sequence selected from the group consisting of: a) a nucleotide sequence including the sequence set forth in SEQ ID NO:3 or a complement thereof; and b) a nucleotide sequence including a fragment of the sequence set forth in SEQ ID NO:3, wherein the sequence initiates transcription in a plant cell; and c) a nucleotide sequence including a sequence having at least 90% sequence identity to the sequence set forth in SEQ ID NO:3, wherein the sequence initiates transcription in the plant cell. In one embodiment, the plant cell is from a dicot. In another embodiment, the heterologous nucleotide sequence encodes a gene product that confers herbicide, salt, cold, drought, pathogen, or insect resistance or the gene product is of agricultural interest.

In another aspect, the invention features a method for selectively expressing a nucleotide sequence in a fruit or ovary, the method including introducing into a plant cell a DNA construct, and regenerating a transformed plant from the plant cell, the DNA construct including a promoter and a heterologous nucleotide sequence operably linked to the promoter, wherein the promoter includes a nucleotide sequence selected from the group consisting of: a) a nucleotide sequence including the sequence set forth in SEQ ID NO:3 or a complement thereof; and b) a nucleotide sequence including a fragment of the sequence set forth in SEQ ID NO:3, wherein the sequence initiates transcription in a plant cell; and c) a nucleotide sequence including a sequence having at least 90% sequence identity to the sequence set forth in SEQ ID NO:3, wherein the sequence initiates transcription in the plant cell. In one embodiment, expression of the heterologous nucleotide sequence alters the phenotype of the fruit or ovary. In another embodiment, the heterologous nucleotide sequence encodes a gene product that confers herbicide, salt, cold, drought, pathogen, or insect resistance or the gene product is of agricultural interest.

In yet another aspect, the invention features a method of preparing food or feed including a) providing any of the aforementioned transgenic plants; and b) preparing food or feed from the plant or a part thereof.

In the context of this disclosure, the term “regulatory element” also refers to a sequence of DNA, usually, but not always, upstream (5′) to the coding sequence of a structural gene, which includes sequences which control the expression of the coding region by providing the recognition for RNA polymerase and/or other factors required for transcription to start at a particular site. An example of a regulatory element that provides for the recognition for RNA polymerase or other transcriptional factors to ensure initiation at a particular site is a promoter element. A promoter element includes a core promoter element, responsible for the initiation of transcription, as well as other regulatory elements (as discussed herein) that modify gene expression. It is to be understood that nucleotide sequences, located within introns, or 3′ of the coding region sequence may also contribute to the regulation of expression of a coding region of interest.

A regulatory element may also include those elements located downstream (3′) to the site of transcription initiation, or within transcribed regions, or both. In the context of this disclosure, a posttranscriptional regulatory element may include elements that are active following transcription initiation, for example translational and transcriptional enhancers, translational and transcriptional repressors, and mRNA stability determinants.

The regulatory elements, or fragments thereof, may be operatively associated with heterologous regulatory elements or promoters in order to modulate the activity of the heterologous regulatory element. Such modulation includes enhancing or repressing transcriptional activity of the heterologous regulatory element, modulating post-transcriptional events, or both enhancing or repressing transcriptional activity of the heterologous regulatory element and modulating post-transcriptional events. For example, one or more regulatory elements, or fragments thereof, of the embodiments may be operatively associated with constitutive, inducible, or tissue preferred promoters or fragments thereof, to modulate the activity of such promoters within desired tissues within plant cells.

The cherry tissue-preferred or tissue-specific promoter sequences of the embodiments, when assembled within a DNA construct such that the promoter is operably linked to a nucleotide sequence of interest, enable expression of the nucleotide sequence in the cells of a plant stably transformed with this DNA construct. The term “operably linked” is intended to mean that the transcription or translation of the heterologous nucleotide sequence is under the influence of the promoter sequence. “Operably linked” is also intended to mean the joining of two nucleotide sequences such that the coding sequence of each DNA fragment remain in the proper reading frame. In this manner, the nucleotide sequences for the promoters described herein are provided in DNA constructs along with the nucleotide sequence of interest, typically a heterologous nucleotide sequence, for expression in the plant of interest. The term “heterologous nucleotide sequence” is intended to mean a sequence that is not naturally operably linked with the promoter sequence. While this nucleotide sequence is heterologous to the promoter sequence, it may be homologous, or native; or heterologous, or foreign, to the plant host. It is recognized that the promoters described herein may be used with their native coding sequences to increase or decrease expression, thereby resulting in a change in phenotype of the transformed plant.

Modifications of the isolated promoter sequences described herein can provide for a range of expression of the heterologous nucleotide sequence. Fragments and variants of the cherry promoter sequences described herein are also encompassed. A “fragment” is intended to mean a portion of the promoter sequence. Fragments of a promoter sequence may retain biological activity and hence encompass fragments capable of driving tissue-preferred expression of an operably linked nucleotide sequence. Thus, for example, less than the entire promoter sequence disclosed herein may be utilized to drive expression of an operably linked nucleotide sequence of interest, such as a nucleotide sequence encoding a heterologous protein. Methods known in the art are useful to determine whether such fragments decrease or increase expression levels or alter the nature of expression, i.e., constitutive or inducible expression. Alternatively, fragments of a promoter nucleotide sequence that are useful as hybridization probes, such as described below, generally do not retain this regulatory activity. Thus, fragments of a nucleotide sequence may range from at least 20 nucleotides, 50 nucleotides, 100 nucleotides, 250 nucleotides, 300 nucleotides, 400 nucleotides, up to 500 nucleotides, up to 600 nucleotides, up to 700 nucleotides, up to 800 nucleotides, up to 900 nucleotides, and even up to 1,000 nucleotides or up to the full-length of the nucleotide sequences disclosed herein.

Thus, a fragment of the cherry promoter nucleotide sequence may encode a biologically active portion of the cherry promoter or it may be a fragment that can be used as a hybridization probe or PCR primer using methods disclosed below. A biologically active portion of the maize cherry promoter can be prepared by isolating a portion of one of the maize cherry promoter nucleotide sequences and assessing the activity of that portion of the maize cherry promoter. Nucleic acid molecules that are fragments of a promoter nucleotide sequence include at least 15, 20, 25, 30, 35, 40, 45, 50, 75, 100, 325, 350, 375, 400, 425, 450, 500, 550, 600, 650, 700, 800, 900, 1000 or up to the number of nucleotides present in the full-length promoter nucleotide sequence disclosed herein, e.g. nucleotides for SEQ ID NO:1, SEQ ID NO:2, SEQ ID NO:3, SEQ ID NO:4, or SEQ ID NO:5.

The nucleotides of such fragments will usually comprise the TATA recognition sequence of the particular promoter sequence. Such fragments may be obtained by use of restriction enzymes to cleave the naturally occurring promoter nucleotide sequence disclosed herein; by synthesizing a nucleotide sequence from the naturally occurring sequence of the promoter DNA sequence; or may be obtained through the use of PCR technology. Variants of these promoter fragments, such as those resulting from site-directed mutagenesis and DNA “shuffling”, are also encompassed by the compositions.

An “analogue” of the regulatory elements of the embodiments includes any substitution, deletion, or addition to the sequence of a regulatory element provided that said analogue maintains at least one regulatory property associated with the activity of the regulatory element of the embodiments. Such properties include directing organ or tissue preference, or a combination thereof, or temporal activity, or developmental activity, or a combination thereof.

The term “variants” is intended to mean sequences having substantial similarity with a promoter sequence disclosed herein. For nucleotide sequences, naturally occurring variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis. Generally, variants of a particular nucleotide sequence will have at least 75%, 80%, 85%, 90%, 91%, 92%, 93%, 94%, to 95%, 96%, 97%, 98%, 99% or more sequence identity to that particular nucleotide sequence as determined by sequence alignment programs described elsewhere herein using default parameters (e.g., SEQ ID NO:1 or a portion thereof; SEQ ID NO:2 or a portion thereof; or SEQ ID NO:3 or a portion thereof; or SEQ ID NO:4 or a portion thereof; or SEQ ID NO:5 or a portion thereof). Biologically active variants are also encompassed. Biologically active variants include, for example, the native promoter sequence having one or more nucleotide substitutions, deletions, or insertions. Promoter activity may be measured by using techniques such as Northern blot analysis, reporter activity measurements taken from transcriptional fusions according to methods well known in the art.

Methods for mutagenesis and nucleotide sequence alterations are also well known in the art.

Variant promoter nucleotide sequences also encompass sequences derived from a mutagenic and recombinant procedure such as DNA shuffling. With such a procedure, one or more different promoter sequences can be manipulated to create a new promoter possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are well known in the art.

The nucleotide sequences described herein can be used to isolate corresponding sequences from other organisms, particularly other plants. In this manner, methods such as PCR, hybridization, and the like can be used to identify such sequences based on their sequence homology to the sequence set forth herein. Sequences isolated based on their sequence identity to the entire maize cherry promoter sequence set forth herein or to fragments thereof are encompassed. The promoter regions of the embodiments may be isolated from any plant, including, but not limited to cherry, almonds, peaches, plums, apricots, and bird cherries. Other exemplary plants include corn (Zea mays), Brassica (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solarium tuberosum), tomato (Lycoperscion esculentum), peanuts (Arachis hypogaea), cotton (Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), oats, barley, vegetables, ornamentals, and conifers. Plants also include corn, soybean, sunflower, safflower, tomato, or canola, wheat, barley, rye, alfalfa, and sorghum.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the cherry promoter sequences. Methods for preparation of probes for hybridization and for construction of cDNA and genomic libraries are generally known in the art.

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing).

Thus, isolated sequences that have tissue-preferred promoter activity and which hybridize under stringent conditions to the cherry promoter sequences disclosed herein, or to fragments thereof, are encompassed.

In general, sequences that have promoter activity and hybridize to the promoter sequences disclosed herein will be at least 40% to 50% homologous, about 60% to 70% homologous, and even about 80%, 85%, 90%, 95% to 98% homologous or more with the disclosed sequences. That is, the sequence similarity of sequences may range, sharing at least about 40% to 50%, about 60% to 70%, and even about 80%, 85%, 90%, 95% to 98% sequence similarity.

Computer implementations of mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0); the ALIGN PLUS program (Version 3.0, copyright 1997): and GAP, BESTFIT, BLAST, FASTA, agnd TFASTA in the Wisconsin Genetics Software Package of Genetics Computer Group, Version 10 (available from Accelrys, 9685 Scranton Road, San Diego, Calif., 92121 , USA). The scoring matrix used in Version 10 of the Wisconsin Genetics Software Package is BLOSUM62 (see Henikoff and Henikoff (1989) Proc. Natl. Acad. Sci. USA 89:10915).

The cherry promoter sequence disclosed herein, as well as variants and fragments thereof, are useful for genetic engineering of plants, e.g., for the production of a transformed or transgenic plant, to express a phenotype of interest. As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations.

The heterologous polynucleotide may be integrated into the genome alone or as part of a recombinant DNA construct. It is to be understood that as used herein the term “transgenic” includes any cell, cell line, callus, tissue, plant part, or plant the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

A transgenic plant is produced by transformation of plant cells with a heterologous DNA construct, including a nucleic acid DNA construct that comprises a transgene of interest, the regeneration of a population of plants resulting from the insertion of the transgene into the genome of the plant, and selection of a particular plant characterized by insertion into a particular genome location.

As used herein, the term “plant” includes reference to whole plants, plant organs (e.g., leaves, stems, roots, etc.), seeds, plant cells, and progeny of same. Parts of transgenic plants are to be understood within the scope of the embodiments to comprise, for example, plant cells, protoplasts, tissues, callus, embryos as well as flowers, stems, fruits, ovules, leaves, or roots originating in transgenic plants or their progeny previously transformed with a DNA molecule of the invention, and therefore consisting at least in part of transgenic cells.

As used herein, the term “plant cell” includes, without limitation, seeds suspension cultures, embryos, mehstematic regions, callus tissue, leaves, roots, shoots, gametophytes, sporophytes, pollen, and microspores. The class of plants that can be used in the methods of the embodiments is generally as broad as the class of higher plants amenable to transformation techniques, including both dicotyledonous and monocotyledonous plants.

The promoter sequences and methods disclosed herein are useful in regulating expression of any heterologous nucleotide sequence in a host plant. Thus, the heterologous nucleotide sequence operably linked to the promoters disclosed herein may be a structural gene encoding a protein of interest.

It is recognized that any gene of interest can be operably linked to the promoter sequences disclosed herein and expressed in plant tissues.

A DNA construct comprising one of these genes of interest can be used with transformation techniques, such as those described below, to create disease or insect resistance in susceptible plant phenotypes or to modulate fruit ripening or other agricultural traits in resistant plant phenotypes. Accordingly, this disclosure encompasses methods that are directed to protecting plants against fungal pathogens, bacteria, viruses, nematodes, and insects.

Disease resistance and insect resistance genes such as lysozymes, cecropins, maganins, or thionins for antibacterial protection, or the pathogenesis-related (PR) proteins such as glucanases and chitinases for anti-fungal protection, or Bacillus thuringiensis endotoxins, protease inhibitors, collagenases, lectins, and glycosidases for controlling nematodes or insects are all examples of useful gene products.

Pathogens include, but are not limited to, viruses or viroids, bacteria, insects, nematodes, or fungi. Viruses include tobacco or cucumber mosaic virus, hngspot virus, necrosis virus, or maize dwarf mosaic virus. Nematodes include parasitic nematodes such as root knot, cyst, and lesion nematodes.

Genes encoding disease resistance traits include avirulence (avr) and disease resistance (R) genes. Insect resistance genes may encode resistance to such as rootworm, cutworm, European corn borer, and the like. Such genes include, for example, Bacillus thuringiensis toxic protein genes.

Herbicide resistance traits may be introduced into plants by genes coding for resistance to herbicides.

Agriculturally useful commercial traits can also be encoded on a gene or genes that could increase for example, starch for ethanol production, or provide expression of proteins.

Agronomically important traits that affect quality of fruit, such as levels and types of oils, saturated and unsaturated, quality and quantity of essential amino acids, levels of cellulose, starch, and protein content can be genetically altered using the methods disclosed herein.

Examples of other applicable genes and their associated phenotype include the gene that encodes viral coat protein and/or RNA, or other viral or plant genes that confer viral resistance; genes that confer fungal resistance; genes that confer insect resistance; genes that promote yield improvement; and genes that provide for resistance to stress, such as dehydration resulting from heat and salinity, toxic metal or trace elements.

RNAi refers to a series of related techniques well known in the art to reduce or silence the expression of genes. The cherry fragments or variants disclosed herein, may be used to drive expression of constructs that will result in RNA interference including microRNAs and siRNAs.

The heterologous nucleotide sequence operably linked to the cherry promoter and related promoter sequences disclosed herein may be an antisense sequence for a targeted gene. By “antisense DNA nucleotide sequence” is meant a sequence that is in inverse orientation to the 5′-to-3′ normal orientation of that nucleotide sequence. When delivered into a plant cell, expression of the antisense DNA sequence prevents normal expression of the DNA nucleotide sequence for the targeted gene. The antisense nucleotide sequence encodes an RNA transcript that is complementary to and capable of hybridizing to the endogenous messenger RNA (mRNA) produced by transcription of the DNA nucleotide sequence for the targeted gene. In this case, production of the native protein encoded by the targeted gene is inhibited to achieve a desired phenotypic response. Modifications of the antisense sequences may be made as long as the sequences hybridize to and interfere with expression of the corresponding mRNA. In this manner, antisense constructions having at least 70%, 80%, or 85% or more sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used. Thus, the promoter sequences disclosed herein may be operably linked to antisense DNA sequences to reduce or inhibit expression of a native protein in selected plant tissues.

In one embodiment, DNA constructs will comprise a transcriptional initiation region comprising one of the promoter nucleotide sequences disclosed herein, or variants or fragments thereof, operably linked to a heterologous nucleotide sequence whose expression is to be controlled by the tissue-preferred promoter of the embodiments. Such a DNA construct is provided with a plurality of restriction sites for insertion of the nucleotide sequence to be under the transcriptional regulation of the regulatory regions. The DNA construct may additionally contain selectable marker genes.

The DNA construct will include in the 5′-3′ direction of transcription, a transcriptional initiation region (i.e., a tissue-preferred promoter of the embodiments), translational initiation region, a heterologous nucleotide sequence of interest, a translational termination region and, optionally, a transcriptional termination region functional in the host organism. The regulatory regions (i.e., promoters, transcriptional regulatory regions, and translational termination regions) and/or the polynucleotide of the embodiments may be native/analogous to the host cell or to each other. Alternatively, the regulatory regions and/or the polynucleotide of the embodiments may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The optionally included termination region may be native with the transcriptional initiation region, may be native with the operably linked polynucleotide of interest, may be native with the plant host, or may be derived from another source (i.e., foreign or heterologous) to the promoter, the polynucleotide of interest, the host, or any combination thereof. Such termination regions are well known in the art.

The DNA construct comprising a promoter sequence of the embodiments operably linked to a heterologous nucleotide sequence may also contain at least one additional nucleotide sequence for a gene to be cotransformed into the organism. Alternatively, the additional sequence(s) can be provided on another DNA construct.

Where appropriate, the heterologous nucleotide sequence whose expression is to be under the control of the tissue-preferred promoter sequence of the embodiments and any additional nucleotide sequence(s) may be optimized for increased expression in the transformed plant. That is, these nucleotide sequences can be synthesized using plant preferred codons for improved expression.

The DNA constructs may additionally contain 5′ leader sequences. Such leader sequences can act to enhance translation.

The DNA constructs of the embodiments can also include further enhancers, either translation or transcription enhancers, as may be required. These enhancer regions are well known to persons skilled in the art, and can include the ATG initiation codon and adjacent sequences. The initiation codon must be in phase with the reading frame of the coding sequence to ensure translation of the entire sequence. The translation control signals and initiation codons can be from a variety of origins, both natural and synthetic. Translational initiation regions may be provided from the source of the transcriptional initiation region, or from the structural gene. The sequence can also be derived from the regulatory element selected to express the gene, and can be specifically modified so as to increase translation of the mRNA. It is recognized that to increase transcription levels enhancers may be utilized in combination with the promoter regions of the disclosure. Enhancers are known in the art and include the SV40 enhancer region, the 35S enhancer element, and the like.

In preparing the DNA construct, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites. Restriction sites may be added or removed, superfluous DNA may be removed, or other modifications of the like may be made to the sequences of the embodiments. For this purpose, in vitro mutagenesis, primer repair, restriction, annealing, re-substitutions, for example, transitions and transversions, may be involved.

Reporter genes or selectable marker genes may be included in the DNA constructs. Examples of suitable reporter genes known in the art are well known such as those described herein.

Selectable marker genes for selection of transformed cells or tissues can include genes that confer antibiotic resistance or resistance to herbicides.

The nucleic acid molecules of the embodiments are useful in methods directed to expressing a nucleotide sequence in a plant. This may be accomplished by transforming a plant cell of interest with a DNA construct comprising a promoter identified herein, operably linked to a heterologous nucleotide sequence, and regenerating a stably transformed plant from said plant cell. The methods of the embodiments are also directed to selectively expressing a nucleotide sequence in a plant tissue. Those methods comprise transforming a plant cell with a DNA construct comprising a promoter identified herein that initiates tissue-preferred transcription in a plant cell, operably linked to a heterologous nucleotide sequence, and regenerating a transformed plant from said plant cell.

The DNA construct comprising the particular promoter sequence of the embodiments operably linked to a nucleotide sequence of interest can be used to transform any plant. In this manner, genetically modified, i.e. transgenic or transformed, plants, plant cells, plant tissue, seed, root, and the like can be obtained.

Plant species suitable for the embodiments include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solarium tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the embodiments include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis). Other plants may be crop plants (for example, corn, alfalfa, sunflower, Brassica, soybean, cotton, safflower, peanut, sorghum, wheat, millet, tomato, and tobacco.

Exemplary useful cherry plants include, without limitation, Prunus alabamensis C. Mohr—Alabama cherry; Prunus alaica (Pojark.) Gilli; Prunus apetala (Siebold & Zucc.) Franch. & Say.—Clove cherry; Prunus avium (L.) L.—Wild cherry, Sweet cherry, Mazzard or Gean; Prunus campanulata Maxim.—Taiwan cherry, Formosan cherry or Bell-flowered cherry; Prunus canescens Bois.—Greyleaf cherry; Prunus caroliniana Aiton—Carolina laurel cherry or Laurel cherry; Prunus cerasoides D. Don.—Wild Himalayan cherry; Prunus cerasus L.—Sour cherry; Prunus cistena Koehne—Purpleleaf sand cherry; Prunus clarofolia C. K. Schneid.; Prunus concinna Koehne; Prunus conradinae Koehne; Prunus cornuta (Wall. ex Royle) Steud.—Himalayan bird cherry; Prunus cuthbertii Small—Cuthbert cherry; Prunus cyclamina Koehne—Cyclamen cherry or Chinese flowering cherry; Prunus dawyckensis Sealy—Dawyck cherry; Prunus dielsiana C. K. Schneid.—Tailed-leaf cherry; Prunus emarginata (Douglas ex Hook.) Walp.—Oregon cherry or Bitter cherry; Prunus eminens Beck—German: mittlere Weichsel (Semi-sour cherry); Prunus fruticosa Pall.—European dwarf cherry, Dwarf cherry, Mongolian cherry or Steppe cherry; Prunus glandulifolia Rupr. & Maxim.; Prunus gondouinii (Poit. & Turpin) Rehder—Duke cherry; Prunus grayana Maxim.—Japanese bird cherry or Gray's bird cherry; Prunus himalaica Kitam.; Prunus humilis Bunge—Chinese plum-cherry or Humble bush cherry; Prunus ilicifolia (Nutt. ex Hook. & Am.) Walp.—Hollyleaf cherry, Evergreen cherry, Holly-leaved cherry or Islay; Prunus incisa Thunb.—Fuji cherry; Prunus jamasakura Siebold ex Koidz.—Japanese mountain cherry or Japanese hill cherry; Prunus japonica Thunb.—Korean cherry; Prunus juddii E. S. Anderson; Prunus laurocerasus L.—Cherry laurel or English laurel; Prunus leveilleana Koehne; Prunus litigiosa C. K. Schneid.; Prunus lusitanica L.—Portugal laurel; Prunus lyonii (Eastw.) Sarg.—Catalina Island cherry; Prunus maackii Rupr.—Manchurian cherry or Amur chokecherry; Prunus mahaleb L.—Saint Lucie cherry, Rock cherry, Perfumed cherry or Mahaleb cherry; Prunus maximowiczii Rupr.—Miyama cherry or Korean cherry; Prunus meyeri Rehder; Prunus myrtifolia (L.) Urb.—West Indian cherry; Prunus nepaulensis (Ser.) Steud.—Nepal bird cherry; Prunus nipponica Matsum.—Takane cherry, Peak cherry or Japanese Alpine cherry; Prunus occidentalis Sw.—Western cherry laurel; Prunus padus L.—Bird cherry or European bird cherry; Prunus pensylvanica L.f.—Pin cherry, Fire cherry, or Wild red cherry; Prunus pilosiuscula (C. K. Schneid.) Koehne; Prunus pleiocerasus Koehne; Prunus pleuradenia Griseb.—Antilles cherry; Prunus prostrata Labill.—Mountain cherry, Rock cherry, Spreading cherry or Prostrate cherry; Prunus pseudocerasus Lindl.—Chinese sour cherry or False cherry; Prunus pumila L.—Sand cherry; Prunus rufa Wall ex Hook.f.—Himalayan cherry; Prunus salicifolia Kunth.—Capulin, Singapore cherry or Tropic cherry; Prunus sargentii Rehder—Sargent's cherry or Ezo Mountain cherry; Prunus schmittii Rehder; Prunus serotina Ehrh.—Black cherry; Prunus serrula Franch.—Paperbark cherry, Birch bark cherry or Tibetan cherry; Prunus serrulata Lindl.—Japanese cherry, Hill cherry, Oriental cherry or East Asian cherry; Prunus setulosa Batalin; Prunus sieboldii (Carriere) Wittm.; Prunus speciosa (Koidz.) Ingram—Oshima cherry;

Prunus ssiori Schmidt-Hokkaido bird cherry; Prunus stipulacea Maxim.; Prunus subhirtella Miq.—Higan cherry or Spring cherry; Prunus takasagomontana Sasaki; Prunus takesimensis Nakai—Takeshima flowering cherry; Prunus tomentosa Thunb.—Nanking cherry, Manchu cherry, Downy cherry, Shanghai cherry, Ando cherry, Mountain cherry, Chinese dwarf cherry, Chinese bush cherry or Hansen's bush cherry; Prunus trichostoma Koehne; Prunus verecunda (Koidz.) Koehne—Korean mountain cherry; Prunus virginiana L.—Chokecherry; and Prunus x yedoensis Matsum.—Yoshino cherry or Tokyo cherry.

By “vector” is meant to a DNA molecule such as a plasmid, cosmid, or bacterial phage for introducing a nucleotide construct, for example, a DNA construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance, or ampicillin resistance.

The methods of the embodiments involve introducing a nucleotide construct into a plant. The term “introducing” is used herein to mean presenting to the plant the nucleotide construct in such a manner that the construct gains access to the interior of a cell of the plant. The methods of the embodiments do not depend on a particular method for introducing a nucleotide construct to a plant, only that the nucleotide construct gains access to the interior of at least one cell of the plant. Methods for introducing nucleotide constructs into plants are known in the art including, but not limited to, stable transformation methods, transient transformation methods, and virus-mediated methods.

By “stable transformation” is intended that the nucleotide construct introduced into a plant integrates into the genome of the plant and is capable of being inherited by progeny thereof.

By “transient transformation” is intended that a nucleotide construct introduced into a plant does not integrate into the genome of the plant. The nucleotide constructs of the embodiments may be introduced into plants by contacting plants with a virus or viral nucleic acids. Generally, such methods involve incorporating a nucleotide construct of the embodiments within a viral DNA or RNA molecule. Methods for introducing nucleotide constructs into plants and expressing a protein encoded therein, involving viral DNA or RNA molecules, are known in the art.

Transformation protocols as well as protocols for introducing nucleotide sequences into plants may vary depending on the type of plant or plant cell targeted for transformation. Suitable methods of introducing nucleotide sequences into plant cells and subsequent insertion into the plant genome are well known in the art.

The cells that have been transformed may be grown into plants in accordance with conventional ways.

The embodiments provide compositions for screening compounds that modulate expression within selected tissues of embryos and plants. The vectors, cells, and plants can be used for screening candidate molecules for agonists and antagonists of the cherry promoter. For example, a reporter gene can be operably linked to a cherry promoter and expressed as a transgene in a plant. Compounds to be tested are added and reporter gene expression is measured to determine the effect on promoter activity.

A 607 bp (SEQ ID NO:2 or SEQ ID NO:4 which includes a transcription start site) and a 929 bp (SEQ ID NO:3 or SEQ ID NO:5 which includes a transcription start site) promoter section of a fruit and ripening specific expansin gene were cloned from cherries (Prunus cerasus). Chimeric promoter-GUS gene constructs were made and introduced by agroinjection into tomatoes. Histochemical GUS staining and enzyme activity measurement showed that both promoter constructs drove the expression of the GUS gene at an equal or higher rate than the tomato E8 promoter. The invention is unique by making available specific promoter constructs that may be used universally among fruit bearing plants to modify their ripening events. Presently a Cauliflower Mosaic Virus 35S promoter is used to silence or up-regulate gene expression in plants. The drawback of this promoter is that it is expressing genes constitutively in every organ of a plant all the times. The cherry expansin promoter constructs, being both organ and event specific, do not have this feature and permit an intelligent approach to interfere with ripening specific processes in fruits.

Most, if not all genetically modified plants presently used commercially have been created using the Cauliflower Mosaic Virus 35S promoter. While the CaMV-35S promoter can be used for improving yield, disease resistance and the vitamin content of plants, it is not desired for the up or down regulation of fruit ripening. The CaMV 35S promoter is a constitutive promoter that expresses the gene constructs all of the time in every organ. To modify fruit ripening, a fruit and ripening specific promoter is needed that expresses the introduced genes only in fruits and only during the ripening phase. The cherry expansin promoter, being both organ and event specific, has this feature and permits an intelligent approach to interfere with ripening specific processes in fruits.

Cherry expansin promoter is both fruit and ripening specific. This means that the promoter will express only the genes that are activated in fruits only, and only during the ripening process. The cherry expansin gene promoter, when introduced into green tomato fruits, expressed the Gus gene only during the ripening process, and its expression was considerably stronger than that of the tomato E8 promoter.

The invention is useful to modify the ripening characteristics of fruits; to extend shelf life of fruits; to improve aroma of fruits by over expressing the genes that control aroma formation; to enhance color formation of fruits, where desirable, by over expressing key pathway genes that control color formation.

Other features and advantages of the invention will be apparent from the following Detailed Description, the drawings, and the claims.

### BRIEF DESCRIPTION OF THE DRAWINGS

The application file contains drawings executed in color (FIGS. 6-12). Copies of this patent or patent application with color drawings will be provided by the Office upon request and payment of the necessary fee.

FIG. 1. SDS-PAGE and Immunoblot analysis of cell wall protein extracts from cherry fruit cv. ‘Montmorency’. (A) SDS-PAGE gel of proteins extracted with 1.5 M NaCl and 0.2 M Na-acetate from the cell walls of cherry fruits from different stages of ripening. (B) immunoblot analysis of the cell wall proteins using the polyclonal LeExp1 antibody. (C) The expansin protein cross-reacted with LeExpl antibody as shown. Mᵣ markers are indicated.

FIG. 2. Tissue specific expression profile of expansin genes. Total RNA was extracted from leaves (L), buds (B), petals (Pt), petioles (P1), and from green (G), pink (P), red (R) and fully ripe (FR) fruit. Specific primers targeting the divergent 3′ UTR region of the expansin genes were designed and used for semi-quantitative RT PCR analysis. 28S and 18S RNA were used for loading control. The PCR products were separated on 1.0% agarose gel and stained with ethidium bromide.

FIG. 3. Nucleotide sequence of the promoter region of the PcExp2 gene. TATA-box, transcription start site, and start codon is shown on the promoter. Ac II elements (GGTTGGT), CCAAT-box, Telo-box (AAACCCTAA) are highlighted. FIG. 4. Schematic description of the PcExp2 gene and the 5′ promoter region cloned from cherry genomic DNA. Promoter region and introns are depicted by solid lines. The three boxes represent the coding sequences (CDS).

FIG. 5. Schematic description of expansin promoter GUS constructs. Fragments of different length from cherry expansin promoter (242 bp, 607 by and 929 bp) and a 1.1 kb of the E8 promoter were cloned by PCR from cherry and tomato genomic DNAs respectively. The promoter fragments were ligated upstream of the GUS reporter gene (uidA) for the transient transformation of the tomato by Agroinfiltration.

FIG. 6. Quantitative measurement of the GUS enzyme activity in the tomatoes agroinjected with the PcPro1 construct.

FIG. 7a. GUS enzyme activity in the tomato fruits agroinjected with the constructs carrying the PcPro2 construct. b Histochemical staining of the tomato fruits agroinjected with the PcPro2 construct.

FIG. 8a. GUS enzyme activity in the tomato fruits agroinjected with the PcPro3 constructs. 8b Histochemical staining of the tomato fruits agroinjected with the PcPro3 constructs.

FIG. 9a. GUS enzyme activity in the tomato fruits agroinjected with the LeProE8 constructs. 9b Histochemical staining of the tomato fruits agroinjected with the LeProE8 constructs.

FIG. 10. Tomato cotyledons cocultivated with Agrobacterium LBA 4404 strain harboring PcPro3 constructs and regenerated—rooted on media containing 100 mg.l⁻¹ kanamycin. Successful transformants were transplanted to soil, transferred to the greenhouse and maintained under a 16 hour photoperiod condition.

FIG. 11. Histochemical staining of non-fruit and fruit tissues obtained from transgenic tomato plants transformed with PcPro3 construct. No GUS staining detected in stems, flowers and leaves. The longest promoter (PcPro3, 929 bp) displayed fruit and ripening specificity. The GUS staining was the highest in fully ripe transgenic tomatoes.

FIG. 12. Relative expression of uidA gene under the control of PcPro3 promoter in leaf, flower, stem, and in the fruits at different developmental stages, with the highest level in the fully ripe fruit as determined by Real—Time PCR (n=3).

FIG. 13. Nucleotide sequence of 607 by promoter (SEQ ID NO:2)

FIG. 14. Nucleotide sequence of 929 by promoter (SEQ ID NO:3)

FIG. 15. Nucleotide sequence of 607 by promoter (SEQ ID NO:4 with a transcription start site.)

FIG. 16. Nucleotide sequence of 6929 by promoter (SEQ ID NO:5 with a transcription start site.)

### DETAILED DESCRIPTION

The following examples are offered by way of illustration and not by way of limitation.

### Results

Fractionation of Cherry Cell Wall Protein Extracts and Detection of the Expansin Proteins during Cherry Fruit Development

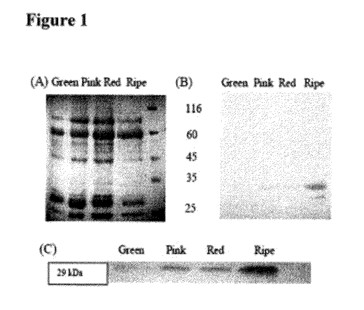

Six major protein bands were detected in the unripe fruits and four of them comprised more than 90% of the total soluble cherry cell wall proteins with the molecular sizes ranging from 18 kDa to 85 kDa (FIG. 1a). In addition to these 6 bands, new protein bands started to appear in the beginning of the ripening stage of fruits indicating the increasing metabolic activity in the fruits during ripening. Protein fractions obtained from ripe cherry fruit cell wall extracts displayed more uniform distribution and there were at least ten major protein bands detected. There was significant accumulation of soluble cherry cell wall proteins during fruit development at the molecular size of 60 kDa and in the range of 25 kDa and 30 kDa (FIG. 1a). When the gels were blotted and the blots probed with a polyclonal tomato anti-expansin antibody (Rose et al. 2000), a 29 kDa cherry cell wall protein band cross reacted with the anti-expansin antibody (FIG. 1b). The blots also showed that the expansin protein band intensity increased in cherry fruits during the development and ripening stages.

Even though cherry fruit maturation can be divided into three distinct stages (Tukey 1936), the presence of expansin activity may be most critical in Stage I (rapid cell division and expansion) and Stage III (final swell). Stage II is thought of as the “retarded growth phase” and obviously there is no exceptional cell wall modification in this period. Western blot data (FIG. 1b) revealed that expansin proteins started to accumulate in the early stage of cherry fruit development which is characterized by rapid cell division and expansion (Stage I) and their expression significantly increased in parallel with development of ripening. The highest amount of expansin proteins were found in the fully ripe and softest cherry fruits.

### Expression of the Expansin Genes During Cherry Maturation and Ripening

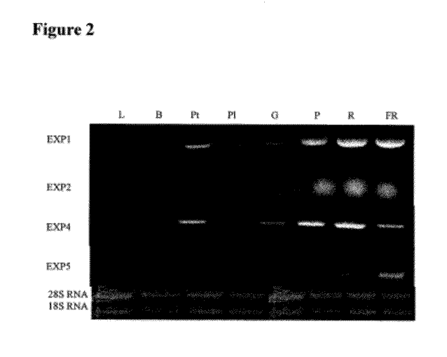

Cherry fruit development and ripening take 50-55 days which can be divided into three distinct stages and involve high level of cell wall metabolism (assembly-disassembly and degradation) (Tukey 1936). To evaluate the potential role of expansin genes in cherry growth and fruit development, mRNA accumulation of the cherry expansin genes has been investigated. Specific primers targeting the highly divergent 3′ untranslated region (UTR) were designed using cherry expansin sequence data published by Gao et al. (2003) and a semi quantitative RT-PCR approach was employed to characterize the transcription of the specific expansin genes. Four expansin genes including PcExp1, PcExp2, PcExp4 and PcExp5 were upregulated during different stages of cherry development (FIG. 2). FIG. 2 shows the expression analysis of four cherry expansin genes on 1.0% (w/v) agarose gel stained with ethidium bromide. Each of the expansins found in cherries displayed different expression pattern and characteristic during fruit development and only two of them were expressed in the vegetative organs (FIG. 2).

The expression of the PcExp1 was very different than the other three expansin genes found in the cherries as shown in FIG. 2. mRNA accumulation of the PcExp1 was detected in leaves, petioles, petals and in fruits throughout growth and ripening, with the highest expression in the fully ripe fruits. PcExp1 transcription was detected first time at green fruit stage and increased gradually during maturation and ripening. The expression of this gene made the sharpest increase that paralleled the final swelling of the cherry fruit.

PcExp1 mRNA was not detected in the buds. Its expression significantly increased after bud braking. The highest PcExp1 mRNA accumulation was detected in petals. There was no detectable PcExp2 mRNA accumulation in vegetative organs such as leaves, petioles, or buds. PcExp2 transcription displayed differential expression in the fruits: PcExp2 was upregulated at the pink fruit stage and high level of PcExp2 mRNA was detected at the later stages of ripening. By contrast to the PcExp1, PcExp2 mRNA was not present at the green fruit developmental stage. PcExp2 was the most abundant expansin gene in cherries displaying ripening specificity. PcExp4 transcription was also detected in fruits throughout fruit development and ripening. However, PcExp4 transcript accumulation peaked before the fruits have reached fully ripe stage and declined afterwards. There was no PcExp4 mRNA accumulation in the vegetative cherry organs such as leaves, petioles, and buds. However, some PcExp4 transcripts were also detected in petals suggesting a specific role for this gene related to petal growth and organ abscission. PcExp5 gene transcription reached detectable levels only after the fruits turned red, and its expression was the highest in the fully ripe fruits. PcExp5 expression was only detected in red stage and ripe fruits and there was no detectable PcExp5 mRNA accumulation in other stages of fruits and vegetative organs. PcExp5 mRNA abundance was very low at the red fruit developmental stage and slightly increased in the final stages of fruit ripening.

Individual members of the cherry expansin family possesses distinct spatial and temporal expression pattern. This data may reflect their specific roles during plant growth and development. According to expression data PcExp1 and PcExp4 may be associated with cell expansion and thus plant growth. Whereas the cumulative accumulation of PcExp2 and PcExp5 mRNAs can be related to cell wall breakdown and softening. However, at this point the role of PcExp1 and PcExp4 in fruit softening can not be discounted.

### Cloning of the Ripening Specific Expansin Gene and its Promoter Fragments

Cherry expansin 2 gene (PcExp2) was identified as one of the expansin genes whose expression lighted up and upregulated during ripening in cherries. Substantial amount of PcExp2 mRNA accumulation was detected in cherries at later stages of fruit maturation. Gene specific primers (PcExp2F and PcExp2R, Table 1) were designed to isolate the PcExp2 gene from cherry genome and amplified DNA fragments have been subsequently sequenced. Sequence data was used to obtain around 1000 by upstream region of the gene. To clone the promoter region of the fruit and ripening specific PcExp2 gene, genomic DNA was isolated from young cherry leaves by the CTAB method (Lodhi et al. 1994) and digested by restriction endonucleases. Genomic DNA fragments were self ligated using T4 DNA ligase (New England Biolabs) and upstream promoter region amplified using inverse PCR (IPCR) approach described by Ochman et al. (1988).

Eventually, a 2596 by sour cherry genomic DNA clone containing the expansin gene (PcExp2) and its putative promoter was obtained. This fragment contains 929 by of the expansin gene upstream region and a 1667 by long open reading frame interspersed with two introns. Sequence comparison of the cherry genomic clone with its cDNA sequence confirmed that the 2.6 kb fragment encoded two introns, three exons and a 929 bp promoter region (FIG. 4). The genomic clone of the PcExp2 gene contains a 74 by 5′ UTR region, one small (156 bp) and one large (440 bp) introns, and a 248 by 3′ UTR region.

The resulting putative promoter sequence was analyzed by Blast against a genome database (NCBI) containing all available plant DNA sequences. No significant similarity was found between the cherry expansin promoter and a number of promoters isolated from many other plant species including tomato, cotton, and Arabidopsis. Interestingly, the only similarity was between the promoter regions of Prunus cerasus expansin 2 (PcExp2) and Pyrus communis expansin 2 (PcoExp2) genes (data not shown).

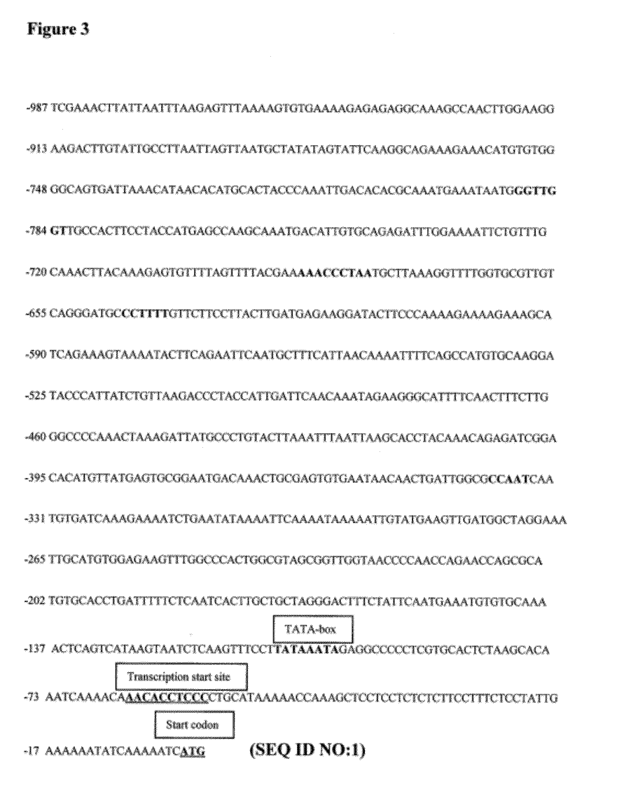

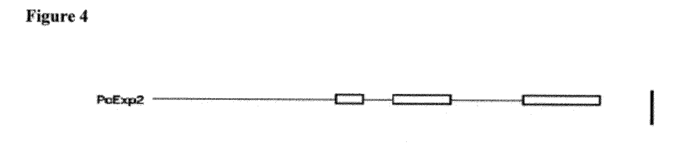

It was reported that 30-40% of the promoter regions are conserved between the orthologous genes isolated from different genera and homology is higher if they are from the same the species (International Chicken Genome Consortium). For instance, two peach 1-aminocyclopropane-1-carboxylic acid oxidase (ACO) promoters exhibited approximately 97% sequence similarity in their first 798 by DNA sequence (Moon and Callahan 2004). There was only a 10-15% similarity between the promoter regions of cherry and pear expansin genes. However, particularly this region may be conserved due to its critical role for the temporal and spatial promotion of the expansin transcription. This 929—nt sequence contains a TATA—box at −38 by relative to the transcription start site (FIG. 3) which is considered as regular feature of eukaryotic promoters (Joshi 1987, Zhu et al. 1995) and consistent with the previously published reports. The upstream region exhibited consensus or highly conserved sequences of CCAAT-boxes, GATA-boxes, pyrimidine box, ethylene responsive element, gibberellin factors, anaerobosis elements, transcriptional activators and cis acting elements conferring tissue specificity.

### A 350 by Region of the PcExp2 Promoter is Required for Gene Expression

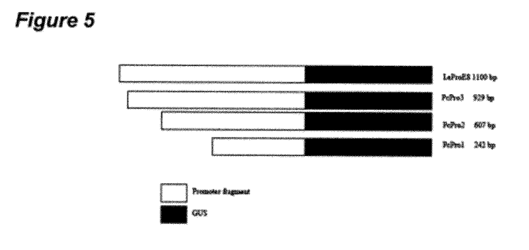

The transcriptional activity of the portions of 5′ flanking region of the expansin promoter was investigated by GUS reporter gene analysis. Truncated series of the expansin promoter and a 1.1 kb of core region of the tomato E8 promoter were amplified by PCR and ligated into the upstream region of the uidA (GUS-β Glucuronidase) gene in the pBI101.1 vector and named PcPro1, PcPro2, PcPro3, LeProE8. The primer pairs that had been used to amplify the expansin 5′ upstream region are shown in Table 1. Schematic description of the truncated promoter series is shown in FIG. 5.

TABLE 1
Primer Sequences

| Primer Name | Sequence |
|---|---|
| Inverse F1 | 5′ CCGAGTGTAAAAGCCAAGAG 3′ |
| Inverse R1 | 5′ CCATTGTTCCCGAGGCATCAC 3′ |
| PcExp1F | 5′ GGCTCTGTCTTTCCAAGTCA 3′ |
| PcExp1R | 5′ CCATAGAAATCCACAAAGTA 3′ |
| PcExp2F | 5′ AGAGCAACACTTACCTCAAT 3′ |
| PcExp2R | 5′ CTTATAAAGCTCTTTGATTT 3′ |
| PcExp4F | 5′ GGATCAGGTTCACAATCAACG 3′ |
| PcExp4R | 5′ GGGTAAAATTGGAAAAACAT 3′ |
| PcExp5F | 5′ CTAGAAGATTAGTATGACTC 3′ |
| PcExp5R | 5′ AGATGACAAGATATTTTATT 3′ |
| 242 bp 5′ | 5′ CCAAGCTTGTATGAAGTTGATGGCTAGG 3′ |
| 607 bp 5′ | 5′ GGAAGCTTAGGGATGCCCTTTTGTTCTT 3′ |
| 929 bp 5′ | 5′ GGAAGCTTTCGAAACTTATTAATTTAA 3′ |
| Promoter R | 5′ GGTCTAGATTATGCAGGGGAGGTGTTTGT 3′ |
| GUS F | 5′ GATCAGCGTTGGTGGGAAAGCGCG 3′ |
| GUS R | 5′ CTACACTCCCCTCACACCGAGGAA 3′ |
| 26S rRNA F | 5′ GCAGCCAAGCCTTCATAGCG 3′ |
| 26S rRNA R | 5′ GTGCGAATCAACGGTTCCTC 3′ |

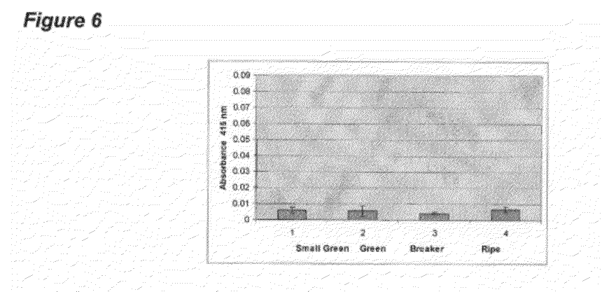

Transgenic tomato fruits agroinjected with constructs containing various size of truncated expansin gene promoters were subjected to histochemical GUS staining and enzyme activity assays. β—Glucuronidase activity was measured in the fruits at different developmental stages of ripening. The average level of β—glucuronidase activity in fruits harboring the constructs carrying expansin promoter fragments was determined spectrophotometrically and displayed in the graphs to show the large changes in GUS activity. According to the results obtained, the construct containing the smallest expansin promoter PcPro1 (242 bp) was not able to direct GUS expression (FIG. 6) in both ripe and unripe fruit.

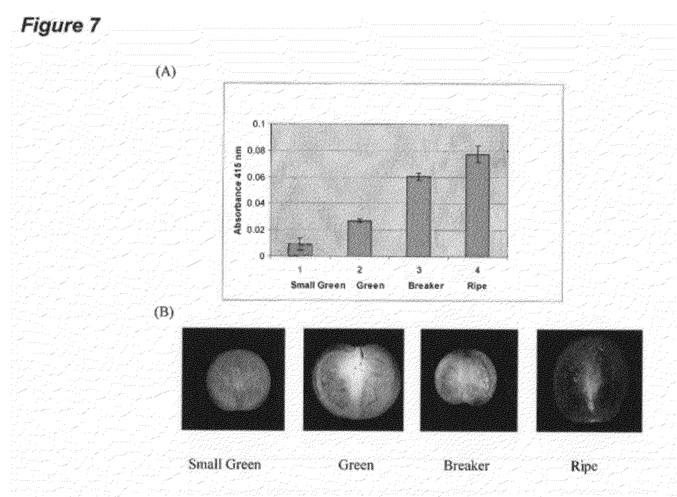

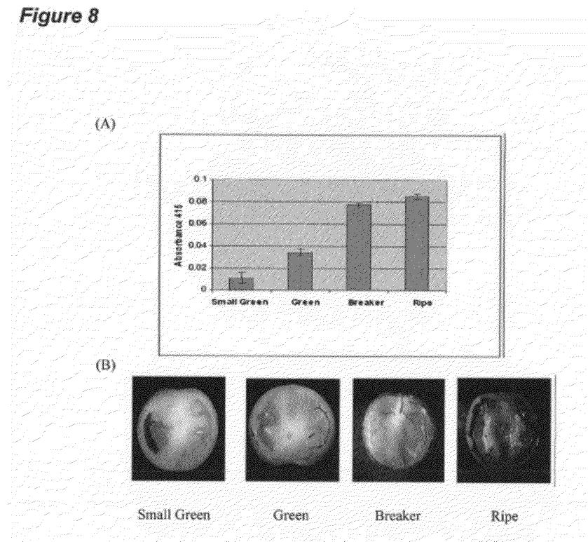

GUS activity above the background level was detected with a PcPro2 promoter construct (607 bp) (FIG. 7a and FIG. 7b). Promoter strength and activity increased in parallel with the length of the 5′ flanking region of the expansin gene and reached the highest level in the PcPro3 promoter (FIG. 8a and FIG. 8b). This data suggested that cis-acting elements acting as positive regulator of expansin expression are most likely located in between the portions of the smallest and medium size promoters and in the distant fragment of the promoter.

Database searches revealed that several cis—acting positive regulators of transcription were missing in the smallest expansin promoter such as ethylene responsive element, gibberellin responsive factor, GATA-box, P-box, Myb-binding site. Therefore an approximately 350 by region between the smallest (242 bp) and medium size (607 bp) promoter established the first significant region conferring promoter activity—strength leading to the conclusion that an expansin promoter as long as around 600 by is sufficient to drive the GUS expression. The transgenic tomatoes injected with the PcPro3 displayed the highest GUS activity in tomatoes from all ripening stages (FIG. 8a and FIG. 8b). These data indicated the presence of additional positive regulators of the expansin promoter located between −600 by and −929 bp.

GUS enzyme activity measurement was carried out in parallel with histochemical analysis to quantitate the ββ-glucuronidase activity. In addition to the small green stage fruits, there was no measurable β-glucuronidase activity in the tomatoes injected with the shortest promoter fragment (FIG. 6) and empty transformation vectors in any of the developmental changes. There was significant activity in tomatoes injected with the medium size (607 bp) promoter. These data also confirmed that an expansin promoter around 0.6 kb size is capable of driving gene expression. Measurable β-glucuronidase activity increased in parallel with fruit ripening and reached the highest level in the fully ripe fruits.

Agroinjected tomato fruits were subjected to the GUS histochemical staining. Typically, GUS staining in fruits from various ripening stages initially was observed in vascular bundles. At the green fruit stage, GUS staining was confined to the vascular bundles and no significant staining was detected in the other parts of the fruit. However, staining was extended to the tomato pericarp and placental tissue in the tomatoes injected with the construct containing the 929 bp length promoter fragment (FIG. 8b).

Tomatoes injected with the longest promoter displayed more intense GUS staining especially in the pericarp, placental tissue and vascular bundles (FIG. 8b). The GUS staining intensity steadily increased in parallel with ripening in agroinjected tomatoes.

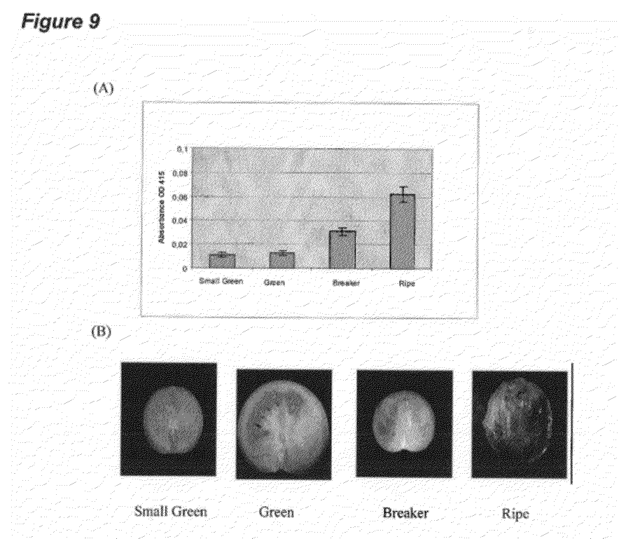

A ripening associated E8 promoter has been characterized in tomatoes (Deikman et al. 1992) and was widely used to drive transgene expression in heterologous plant transformation studies. A 1.1 kb fragment of the “late-expressing” E8 promoter has been used for vaccine expression in ripe fruits (He et al. 2008). The organ and developmental stage specificity of the E8 genes have been shown in tomatoes by GUS reporter gene assays (Montgomery 1993). Therefore a 1.1 kb core region of E8 promoter was fused to the upstream region of uidA gene open reading frame and introduced to the tomato fruits to investigate the expression pattern of another ripening and fruit specific promoter. Fruits agroinjected with the constructs harboring the fragment of the E8 promoter were subjected to histochemical and enzyme activity assays to compare the promoter strength—activity of the E8 promoter and the cherry expansin promoters (FIG. 9a and FIG. 9b). E8 promoter injected unripe tomatoes did not display significant GUS staining (FIG. 9b). However, GUS staining increased to detectable levels after the fruits turned breaker stage and reached the highest level in the fully ripe fruit. The sharpest increase in GUS staining observed in the fully ripe fruits indicated the fruit and ripening specificity of the E8 promoter. GUS staining started to increase at the breaker stage fruits in tomatoes agroinjected with the PcExp-GUS constructs and reached the highest level in the ripe fruits. Both cherry expansin 5′ fragments (PcPro2 and PcPro3) displayed stronger promoter activity in comparison to the E8 promoter (FIG. 7a, FIG. 8a and FIG. 9a). This result makes both expansin promoters useful for transformation studies targeting the improvement of the cherry fruit quality.

### Temporal and Spatial Activity of Cherry Expansin Gene Promoter

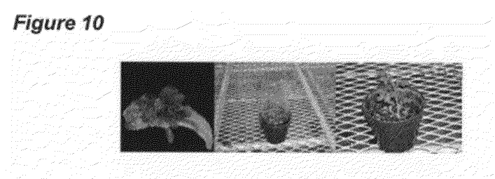

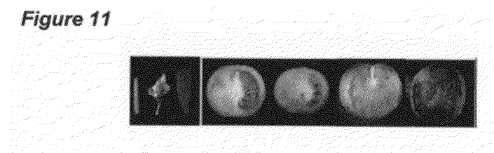

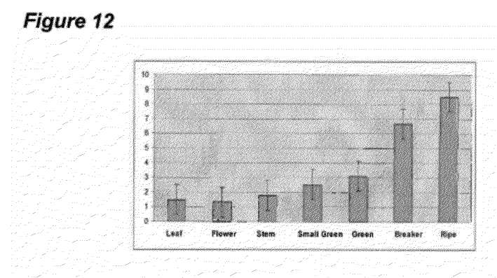

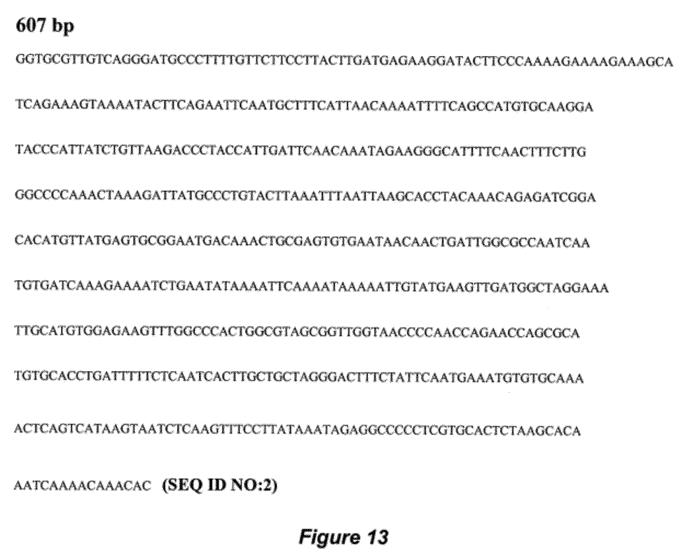

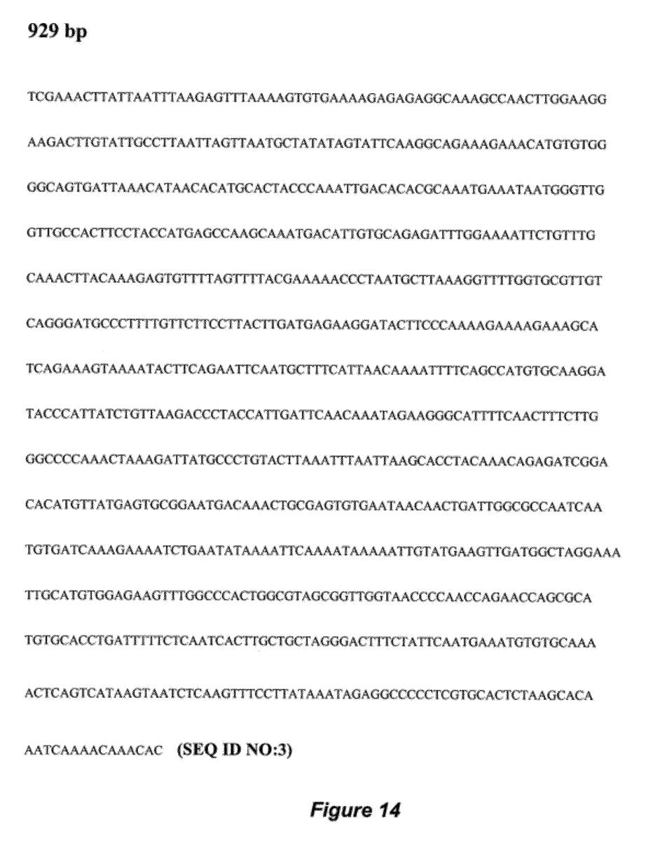

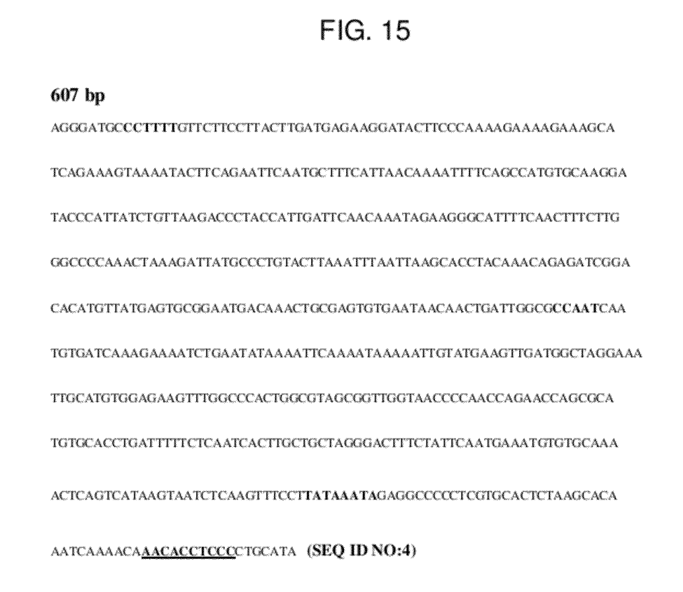

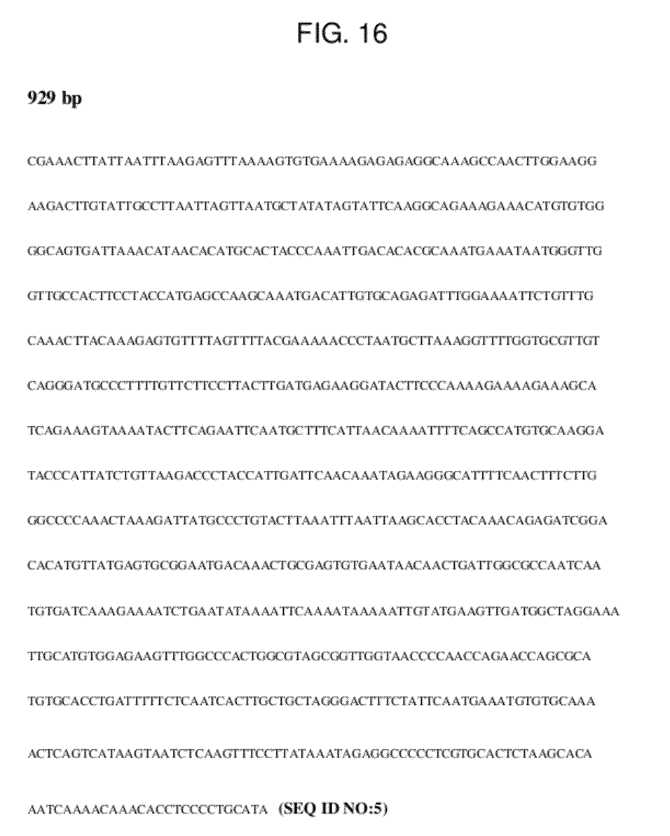

Tomato plants stably transformed with the construct containing the longest insert (929 bp, (FIG. 10)), which was shown to be the strongest among the other promoter fragment. GUS staining and expression were assayed using histochemical staining and qRT-PCR (Real Time PCR) approach both in fruit and non-fruit tissues to investigate the temporal and spatial activity of the promoter. Despite the fact that there was no GUS staining in vegetative and floral tissues including stems, leaves and flowers, GUS histochemical staining significantly increased in tomato fruits during the development of ripening and reached the highest level in fully mature transgenic fruits (FIG. 11). Real-Time PCR was used to determine accumulation of GUS transgene in leaf, flower, stems and fruit tissues and the data normalized against the amount of 26S rRNA (Hangsik M and Callahan A M 2004). In parallel with the results obtained from staining experiments there was no detectable transgene accumulation above background values in vegetative and floral organs however the uidA gene transcription significantly increased in the fruit tissues (FIG. 12). This result also confirmed the controlling ability of cherry expansin promoter on transgene expression tissue and developmental specific manner. Findings obtained from the stable tomato transformation experiments indicated that this promoter is a valuable molecular tool to manipulate plant based food quality traits.

The above-described results were obtained using the following materials and methods.

### Materials and Methods

### Nucleic Acids Extraction

Genomic DNA was extracted from unexpanded young cherry leaves using the method of Lodhi et al (1994). RNA used in this study was obtained from plant tissues by phenol/chloroform extraction according to method described by Lopez-Gomez and Gomez-Lim M A (1992). Total RNA quality and quantity was determined by denaturing formaldehyde gel and using a Spectronic® Genesys™ spectrophotometer respectively.

### Cell Wall Protein Extraction

The extraction method described by Fils-Lycaon et al. (1996) was essentially used to obtain cherry cell wall proteins. Complete™ protease inhibitor cocktail tablets (Roche) were added to the extraction buffer to inhibit protease activity during extraction. Half volume of 20% trichloroacetic acid (TCA) was used to precipitate the cell wall proteins. The pellet was washed three times with acetone and air dried to remove residual acetone. Protein was dissolved in SDS-PAGE sample buffer and stored at −80° C. until used.

### SDS—Polyacrylamide Gel Electrophoresis (SDS-PAGE) and Immunoblotting

Analytical SDS-PAGE was performed in Bio-Rad (Bio-Rad, Hercules, Calif., USA) minigels (Laemmli UK 1970) by using general laboratory techniques (Sambrook et al. 1989). Expansin proteins were detected using polyclonal tomato expansin (LeExp1) antibody diluted 1:1,500 in TBS buffer. The proteins cross-reacting with the expansin antibody were visualized with anti-rabbit antibody conjugated to alkaline phosphatase (Sigma). Nitroblue tetrazolium (NBT) and 5-bromo-4-chloro-3-indolyl phosphate (BCIP) were dissolved in alkaline phosphatase developing buffer and used as substrate for color development.

### Inverse PCR and Isolation of the Expansin Promoter

Expansin gene promoter isolation was accomplished using inverse PCR (IPCR) technique as described by Ochman et al. (1988). Fifteen μg genomic cherry DNA was digested with Taq I restriction endonuclease. Four μl of digested and purified genomic DNA (40 ng) was self ligated to use as template for inverse PCR. PCR primers oriented in the reverse direction of the usual orientation were designed to amplify the flanking region (Table 1).

The PCR reaction profile was: 94° C. for 3 minutes for the initial denaturation step and 35 cycles of 94° C. for 30 seconds, 62° C. for 30 seconds, 70° C. for 4 minutes and followed by one cycle of 70° C. for 15 minute. The PCR products were analyzed on 1.2% agarose gel. The correct size bands were excised from the gel, purified and subsequently sequenced. New primer pairs were designed targeting the putative promoter region and the coding sequence of PcExp2 gene to amplify the upstream region. PCR products were sequenced to confirm nucleotide sequence.

### Tomato Transformation

Constructs harboring different size of PcExp2 promoter fragments were electroporated into Agrobacterium LBA4404 strain and grown on media containing selective antibiotics. The agroinjection procedure described by Orzeaz et al. (2006) was closely followed for the transient gene expression analysis.

Tomatoes stably transformed according to Boyce Thompson Institute (BTI) transformation protocol which is a modified version of the method described by Fillatti et al (1987). Cotyledons were cut into smaller pieces (0.5 cm) and co-cultivated with the Agrobacterium LBA4404 culture harboring the constructs. Successful transformants were selected and rooted on media containing 100 mg l⁻¹ kanamycin and then transferred to the greenhouse. The presence of the construct in the tomato plants was verified by PCR.

Reverse Transcriptase PCR (Semi Quantitative RT-PCR) and Real Time PCR (qRT-PCR)

Total RNA was extracted from different cherry tissues as described above (Lopez-Gomez and Gomez-Lim M A 1992) and treated with RQ1 RNase-Free DNase kit (Promega) to remove contaminating genomic DNA. SuperScript™ III One-Step RT-PCR system with Platinum® Taq DNA Polymerase kit was used for semi quantitative RT-PCR assay according to manufacturer's instructions (Invitrogen, life technologies). Reactions were incubated at 55° C. for 30 minutes for the cDNA synthesis and denatured at 94° C. for 2 minutes prior to PCR amplification. Primers used for RT-PCR analysis are shown in Table 1. The PCR reaction profile was: 34 cycles at 94° C. for 15 seconds, 56° C. for 30 seconds, 68° C. for 1 minute and followed by one cycle of 68° C. for 5 minutes. PCR products were separated on 1.2% agarose gel, visualized and the gels were photographed.

qRT-PCR analysis was carried out by using iScript™ One-step RT-PCR Kit with SYBR® Green (Bio-Rad, Hercules, Calif.) in the iCycler instrument (Bio-Rad). Real Time PCR cycling conditions were 50° C. for 10 minutes (cDNA synthesis), 95° C. for 5 minutes (reverse transcriptase inactivation), 40 cycles of 95° C. for 10 seconds and 56° C. for 30 seconds (primer annealing and extension). Melting point analyses were performed after each run to confirm specificity of the primers and to detect primer-dimers and secondary products. The accumulation of uidA was normalized against the expression of the 26S rRNA (Hangsik M and Callahan AM 2004), primer sequences are shown in Table 1. The qRT-PCR data were analyzed using a comparative ΔΔC_{T} method (Livak K J and Schmittgen T D 2001). The ΔC_{T} was calculated according to the difference between C_{T} of normalizer and target genes and expressed as relative mRNA levels.

### GUS Reporter Gene Constructions

The binary vector, pBI101.1, containing a GUS coding sequence, was used to make reporter gene constructs (Jefferson et al. 1987). Three different sizes of expansin gene promoter fragments (242 bp, 607 bp, and 929 bp) with respect to transcription start site were obtained by PCR amplification using primers ProF1, ProF2, ProF3 together with ProR (Table 1). The E8 promoter was obtained from tomato genomic DNA using primers E8F and E8R (Table 1). HindIII and XbaI restriction enzyme sites were introduced to all the promoter fragments via site directed mutagenesis and cloned into the pBI101 vector double digested with HindIII and XbaI enzymes. Construct fidelity was assured by PCR and sequencing.

### GUS Histochemical Staining and Enzyme Activity Assay

GUS histochemical staining was carried out as described by Jefferson RA (1987). Methanol (20%) was added to the buffer to inhibit background β-glucuronidase activity (Kosugi et al. 1990) and staining was carried out at 37° C. To measure GUS enzyme activity, tomato fruit slices were homogenized in GUS extraction buffer and enzyme activity was determined spectrophotometrically (Jefferson R A 1987). Fifty μl of extract was added to 950 μl of assay buffer containing methanol and mixed thoroughly by vortexing. Reaction tubes were incubated overnight at 37° C. The reaction was stopped by addition of 0.4 ml 2.5 M 2-amino-2-methyl propanediol. Another reaction mixture was prepared and stopped immediately after addition of the GUS extract. Absorbance was measured against a stopped reaction mixture at 415 nm.

### Discussion

Tomato has been successfully used as a model system in order to unravel the underlying mechanisms of climacteric fleshy fruit ripening and thus much of the research has been focused on this fruit. Ethylene coordinates the ripening associated biochemical reactions in climacteric fruits. The majority of the tomato fruit research has been focused on ethylene biosynthesis and signal transduction pathways. There is limited amount of research on non-climacteric fruit ripening and the role of ethylene is not clear. However, there are several reports in the literature implicating ethylene in non climacteric fruit ripening such as pigmentation in citrus (Alonso et al. 1995) and gene expression in strawberries (Tesniere et al. 2004). One recent study reported that ethylene may be involved in the regulation of VvADH2 gene expression in grapes (Vitis vinifera, one of the non-climacteric type fruits) (Tesniere et al. 2004). The ethylene responsive element (ERE) and the anaerobic-responsive-element (ARE) are located in the promoter region of the VvADH2 gene. ERE and ARE elements are known as positive regulators of gene expression (Walker et al. 1987, Chen et al. 1993). ARE elements were identified in the upstream regions of AtADH1 (Arabidopsis thaliana) and LeADH2 (Solanum lycopersicum) genes and their expression was upregulated under low-oxygen stress (Daraselia et al. 1996, Feldbrugee et al. 1994). The role of ERE cis-acting element on the induction of ADH expression was not established yet (Tesniere et al. 2004). However, exogenous ethylene application substantially induced VvADH2 expression. Ethylene data and presence of ERE and ARE elements suggested a role for ethylene in the non climacteric fruit ripening.

An ERE and an ARE element were also found in the cherry expansin gene promoter region. Cherries have been classified as non-climacteric and there is no detectable ethylene burst at the onset of the ripening and ethylene synthesis rate remains very low during the time course of ripening (around 1-2 pmol/kg.hr, data not shown). Ethylene production rate and expansin mRNA accumulation does not correlate well in cherries. Young green cherry fruits produce substantially higher amount of ethylene in comparison to the ripe fruits, while expansin mRNAs are most abundant in fully ripe fruits. Therefore, it does not seem feasible that expansin expression is positively regulated by ethylene in cherries.

Auxin and ethylene regulates the expression of AtExp7 (Arabidopsis thaliana expansin gene) at the root hair formation sites. Reporter gene analyses have been carried out to characterize the promoter region of the AtExp7. Database searches revealed that auxin (ARE) and ethylene responsive elements (ERE) are located in the distal region of the AtExp7 promoter (Cho and Cosgrove 2002). However, removal of these elements did not alter the responsiveness of the promoter to the auxin and ethylene treatments. Deletion analysis indicated that, DOF zinc finger binding domains (AAAG) and a MYB-like (MYBSt1) protein binding site (GGATA) located at the proximal region of the promoter is critical for inducibility of the AtExp7 expression. A second MYBSt1 core motif located at the distal region of the promoter was also found to be associated with AtExp7 expression (promoter activity). In addition to ethylene and gibberellin responsive elements, a series of DOF zinc finger domain and MYB-like protein binding motifs are located on the PcExp2 promoter in a similar pattern found in that of AtExp7 promoter. This region is composed of 371 nucleotides. An internal deletion covering this fragment significantly reduced expansin promoter activity. Interestingly, this fragment corresponds to the only conserved region in between promoter regions of PcoExp2 and PcExp2 genes.

### Cis Acting Elements Found in the Expansin Promoter

Environmental and hormonal signals regulate differential expression of expansin genes and these are present in the upstream regions of expansin genes (Lee et al. 2001). It was found that expansin expression can be regulated by auxin (Catalá et al. 2000, Hutchison et al. 1999), gibberellin (Cho and Kende 1997, Lee and Kende 2001), cytokinin (Malley and Lynn 2000, Wrobel and Yoder 2001) and ethylene (Kim et al. 2001, Vriezen et al. 2000).

Transcription factors recognize and bind to short DNA sequences present at the upstream region of genes (Fickett J W and Hatzigeorgiou E G 1997). DNA binding factors specifically bind to certain sequences and are significant elements of growth and differentiation. PLACE, PlantCare and MatInspector databases were used to identify the cis-acting and hormone responsive elements present at the upstream region of the PcExp2 gene. The elements were found to be critical for the activity and strength of the promoters isolated from the other species.

The cis-acting elements that confer tissue and developmental stage specificity to the expression of the plant genes such as Myb protein binding-site (GGATA-box) (Baranowski et al. 1994), CCAAT-box (Rieping M and Schoffl F. 1992; Hatamochi et al. 1998; Wenkel et al. 2006; Haralampidis et al. 2002), Telomere motif (Telo-box) (Liboz et al. 1991; Curie et al. 1993; Regad et al. 1995; Tremousayque et al. 1999), AC elements (Leyva et al. 1994; Kapoor S and Sugiura M. 1999), GT-1 cis element (Park et al. 2004), and GAGA-box (Sangwan I and O'Brian M R 2002) found to be conserved in the PcExp2 upstream region. These elements were shown to be associated with temporal and spatial regulation of the fruit and organ specific gene expression. Further deletion analyses are necessary to elucidate the role of these elements in the activity and strength of the PcExp2 promoter.

Cherry is a true (ovary-derived) fruit and represent a valuable model to study the underlying mechanisms of softening in non-climacteric fruits (Gao et al. 2003) including the isolation of tissue-ripening specific promoters for the genetic engineering of a variety of fruits. The expansin promoter which is cloned in this study is a potentially valuable molecular tool to manipulate fruit quality factors in both climacteric and non-climacteric fruits including commercially important tomato. The availability of the tissue and development specific promoter can provide an advantage to develop precise molecular tools to interfere with gene expression related to fruit softening. Promoter research holds the promise to precise manipulation of gene expression and thus biological pathways without affecting other metabolic events. Expansin promoter can be utilized to genetically engineer cherries and other crops to produce transgenic fruits that have superior quality and extended shelf life.

### REFERENCES

Alonso J M, Chamaro J, Granell A (1995) Evidence for the involvement of ethylene in the expression of specific RNAs during maturation of the orange, a non-climacteric fruit. Plant Mol Biol 29: 385-390.

Baranowski N, Frohberg C, Prat S, Willmitzer L (1994) A novel DNA binding protein with homology to Myb oncoproteins containing only one repeat can function as a transcriptional activator. EMBO J 13: 5383-5392.

Belfield E J, Ruperti B, Roberts J A, McQueen-Mason S (2005) Changes in expansin activity and gene expression during ethylene-promoted leaflet abscission in Sambucus nigra. J Exp Bot 56:817-823.

Brummell D A, Harpster M H, Civello P M, Palys J M, Bennett A B, Dunsmuir P (1999) Modification of expansin protein abundance in tomato fruit alters softening and cell wall polymer metabolism during ripening. Plant Cell 11:2203-2216.

Brummel D A, Harpster M H (2001) Cell wall metabolism in fruit softening and quality and its manipulation in transgenic plants. Plant Mol Biol 47: 311-340.

Catalá C, Rose J K C, Bennett A B (2000) Auxin-regulated genes encoding cell wall-modifying proteins are expressed during early tomato fruit growth. Plant Physiol 122: 527-534.

Chen A R S, Chase Jr T (1993) Alcohol dehydrogenase 2 and pyruvate decarboxylase induction in ripening and hypoxic tomato fruit. Plant Physiol Biochem 31: 875-885

Cho H T, Kende H (1997) Expression of expansin genes is correlated with growth in deepwater rice. Plant Cell 9:1661-1671.

Cho H-T, Cosgrove D J (2002) Regulation of root hair initiation and expansin gene expression in Arabidopsis. Plant Cell 14: 3237-3253.

Cosgrove D J (2000) Loosening of plant cell walls by expansins. Nature 407:321-326.

Cosgrove D J (1998) Cell wall loosening by expansins. Plant Physiol 118:333-339.

Cosgrove D J, Bedinger P, Durachko DM (1997) Group I allergens of grass pollen as cell wall-loosening agents. PNAS 94:6559-6564.

Curie C, Axelos M, Bardet C, Atanassova R, Chaubet N, Lescure B (1993). Modular organization and developmental activity of an Arabidopsis thaliana eEF1A gene promoter. Mol Gen Genet 238: 428-436.

Daraselia N D, Tarchevskaya S, Narita J O (1996) The promoter for tomato 3-hydroxy-3-methylglutaryl coenzyme A reductase gene has 2 unusual regulatory elements that direct high-level expression. Plant Physiol 112: 727-733.

Davuluri G R, van Tuinen A, Fraser P D, Manfredonia A, Newman R, Burgess D, Brummell D A, King S R, Palys J, Uhlig J, Bramley P M, Pennings H M, Bowler C (2005) Fruit specific RNAi-mediated suppression of DET1 enhances carotenoid and flavonoid content in tomatoes. Nat Biotechnol 23: 890-895.

Davuluri G R, van Tuinen A, Mustilli A C, Manfredonia A, Newman R, Burgess D, Brummell D A, King S R, Palys J, Uhlig J, Bramley P M, Uhlig J, Pennings HM, Bowler C (2004) Manipulation of DET1 expression in tomato results in photomorphogenic phenotypes caused by post-transcriptional gene silencing. 40: 344-354.

Deikman J, Kline R, Fischer R L (1992) Organization of ripening and ethylene regulatory regions in fruit-specific promoter from tomato. Plant Physiol 100: 2013-2017.

Feldbrugge M, Sprenger M, Dinkelbach M, Yazaki K, Harter K, Weisshaar B (1994) Functional analysis of a light-responsive plant bZIP transcriptional regulator. Plant Cell 6: 1607-1621.

Fickett J W, Hatzigeorgiou E G (1997) Eukaryotic promoter recognition. Genome Res 7: 861-878.

Fillatti J J, Kiser J, Rose R, Comai L (1987) Efficient transfer of a glyphosate tolerance gene into tomato using a binary Agrobacterium tumefaciens vector. Biotechnology 5: 726-730.

Fils-Lycaon B R, Wiersma P A, Eastwell K C, Sautiere P (1996) A cherry protein and its gene abundantly expressed in ripening fruit, have been identified as thaumatin-like. Plant Physiol 111: 269-273.

Fry S C, Smith R C, Renwick K F, Martin D J, Hodge S K, Matthews K J (1992) Xyloglucan endotransglycosylase, a new wall-loosening enzyme activity from plants. Biochem J 282: 821-828.

Fry C S (1989) Cellulases, hemicelluloses and auxin-stimulated growth: a possible relationship. Physiologia Plantarum 75: 532-536.

Gao Z, Maurousset L, Lemoine R, Yoo S-D, van Nocker S, Loescher W (2003) Cloning, expression, and characterization of sorbitol transporters from developing sour cherry fruit and leaf sink tissues. Plant Physiol 131: 1566-1575.

Giovannoni J J, DellaPenna D, Bennett A B, Fischer R L (1989) Expression of a chimeric polygalacturonase gene in transgenic rin (ripening inhibitor) tomato fruit results in polyuronide degradation but not fruit softening. Plant Cell 1: 53-63.

Hammond-Kosack K E, Parker J E (2003) Deciphering plant-pathogen communication: fresh perspectives for molecular resistance breeding. Curr Opin Biotechnol 14: 177-193.

Hatamochi A, Golumbek P T, Vanschaftingen E, Decrombrugghe B (1998) A CCAAT DNA-binding factor consisting of 2 different components that are both required for DNA-binding. J Biol Chem 263: 5940-5947.

Hangsik M and Callahan A M (2004) Developmental regulation of peach ACC oxidase gene promoter-GUS fusions in transgenic tomato studies. J Exp Bot 55: 1519-1528.

Haralampidis K, Milioni D, Rigas S, Hatzopoulos P (2002) Combinatorial interaction of cis elements specifies the expression of the Arabidopsis AtHsp90-1 gene. Plant Physiol 129: 1138-1149.

Harrison P E, McQueen-Mason, S J, Manning K (2001) Expression of six expansin genes in relation to extension activity in developing strawberry fruit. J Exp Bot 52:1437-1446.

He Z-M, Jiang X-L, Qi Y, Luo D-Q (2008) Assessment of the utility of the tomato fruit-specific E8 promoter for driving vaccine antigen expression. Genetica 133: 207-214.

Hutchison K W, Singer P B, McInnis S, Diaz-Sala C, Greenwood M S (1999) Expansins are conserved in conifers and expressed in hypocotyls in response to exogenous auxin. Plant Physiol 120: 827-831.

International Chicken Genome Sequencing Consortium (2004) Sequence and comparative analysis of the chicken genome provide unique perspectives on vertebrate evolution. Nature 432: 695-716.

Jefferson R A, Kavanagh T A, Bevan M W (1987) GUS fusions: β-Glucuronidase as a sensitive and versatile gene marker in higher plants: EMBO J 6: 3901-3907.

Jefferson R A (1987) Assaying chimeric genes in plants: the GUS gene fusion system. Plant Mol Biol Rep 5: 387-405.

Joshi C P (1987) An inspection of the domain between putative TATA box and translation start site in 79 plant genes. Nucleic Acid Research 15: 6644-6652.

Kapoor S, Sugiura M (1999) Identification of Two Essential Sequence Elements in the Nonconsensus Type II PatpB-290 Plastid Promoter by Using Plastid Transcription Extracts from Cultured Tobacco BY-2 Cells. Plant Cell 11: 1799-1810.

Kim K Y, Kwon S Y, Lee H S, Hur Y, Bang J W, Kwak S S (2003) A novel oxidative stress-inducible peroxidase promoter from sweet potato: molecular cloning and characterization in transgenic tobacco plants and cultured cells. Plant Mol Biol 51: 831-838.

Kim J H, Cho H T, Kende H (2000) β-Expansins in the semiaquatic ferns Marsilea quadrifolia and Regnellidium diphyllum: evolutionary aspects and physiological role in rachis elongation. Planta 212: 85-92.

Kosugi S, Ohashi Y, Nakajima K, Arai Y (1990) An improved assay for β-glucuronidase in transformed cells: Methanol almost completely supresses a putative endogenous 13-glucuronidase activity. Plant Sci 70: 133-140.

Laemmli U K (1970) Cleavage of structural proteins during the assembly of the head of bacteriophage T4. Nature 227: 680-685.

Lee Y, Kende H (2001) Expression of β-expansins is correlated with elongation of internodes in deepwater rice. Plant Physiol 2001, 127: 645-654.

Lee Y, Choi D, Kende H (2001) Expansins ever-expanding numbers and functions. Curr Opin Plant Biol 4: 527-532.

Leyva A, Liang X, Pintor-Toro J A, Dixon R A, Lamb C J (1992) cis-Element Combinations Determine Phenylalanine Ammonia-Lyase Gene Tissue-Specific Expression Patterns. Plant Cell 4: 263-271.

Liboz T, Bardet C, Le Van Thai A, Axelos M, Lescure B (1991) The four members of the gene family encoding the Arabidopsis thaliana translation elongation factor eEF1A are actively transcribed. Plant Mol Biol 14: 107-110.

Livak K J, Schmittgen T D (2001) Analysis of relative gene expression data using real time quantitative PCR and the 2^{-ΔΔCT} method. Methods 25: 402-408.

Lodhi M A, Ye Guang-Ning, Weeden N F, Reisch B I (1994) A simple and efficient method for DNA extraction from grapevine cultivars, Vitis species and Ampelopsis. Plant Mol Biol Rep 12: 6-13.

Lopez-Gomez R, Gomez-Lim M A (1992) A method for extracting intact RNA from fruits rich in polysaccharides using ripe mango mesocarp. Hortscience 27: 440-442.

Malley R C, Lynn D G (2000) Expansin message regulation in parasitic angiosperms: marking time in development. Plant Cell 12: 1455-1465.

McQueen-Mason S J, Durachko D M, Cosgrove D J (1992) Two endogenous proteins that induce cell wall extension in plants. Plant Cell 4:1425-1433.

Montgomery J, Pollard V, Deikman J, Fischer R L (1993) Positive and negative regulatory regions control the spatial distribution of polygalacturonase transcription in tomato fruit pericarp. Plant Cell 5: 1049-1062.

Moon H, Callahan A M (2004) Developmental regulation of peach ACC oxidase promoter-GUS fusions in transgenic tomato fruits. J Exp Bot 55: 1519-1528.

Nishitani K, Tominaga R (1992) Endo-xyloglucan transferase, a novel class of glycosyltransferase that catalyzes transfer of a segment of xyloglucan molecule to another xyloglucan molecule. J Biol Chem 267: 21058-21064.

Ochman H, Gerber A S, Hard D L (1988) Genetic applications of an inverse polymerase chain reaction. Genetics 120: 621-623.

Park H C, Kim M L, Kang Y H, Jeon J M, Yoo J H, Kim M C, Park C Y, Jeong J C, Moon B C, Lee J H, Yoon H W, Lee S H, Chung W S, Lim C O, Lee S Y, Hong J C, Cho M J (2004) Pathogen- and NaCl-induced expression of the SCaM-4 promoter is mediated in part by a GT-1 box that interacts with a GT-1-like transcription factor. Plant Physiol 135: 2150-2161.

Regad F, HerveÂ C, Marinx O, Bergounioux C, Tremousaygue D, Lescure B (1995) The tef1 box, a ubiquitous cis-acting element involved in the activation of plant genes that are highly expressed in cycling cells. Mol Gen Genet 248:703-711.

Rieping M, Schoffl F (1992) Synergistic effect of upstream sequences, and HSE sequences for enhanced expression of chimeric heat shock genes in transgenic tobacco. Mol Gen Genet 231: 226-232.

Rose J K C, Lee H H, Bennett A B (1997) Expression of a divergent expansin gene is fruit-specific and ripening regulated. PNAS 94:5955-5960.

Rose J K C, Cosgrove D J, Albersheim P, Darvill A G, Bennett A B (2000) Detection of expansin proteins and activity during tomato fruit ontogeny. Plant Physiol 123: 1583 1592.

Sambrook J, Fritsch E F, Maniatis T (1989) Molecular cloning. A laboratory manual, second edition. Cold Spring Harbor Laboratory Press.

Sane A P, Tripathi S K, Nath P (2007) Petal abscission in rose (Rosa bourboniana var Gruss an Teplitz) is associated with the enhanced expression of an alpha expansin gene, RbEXPA1, Plant Science 172:481-487.

Sangwan I, O'Brian M R (2002) Identification of a soybean protein that interacts with GAGA elements dinucleotide repeat DNA. Plant Physiol 129: 1788-1794.

Scherban T Y, Shi J, Durachko D M, Guiltina M J, McQueen-Mason S J, Shieh M, Cosgrove D J (1995) Molecular cloning and sequence analysis of expansins, a highly conserved multigene family of proteins that mediate cell wall extension in plants. PNAS 92: 9245-9249.

Shin Y-J, Hong S-Y, Kwon T-H, Jang Y-S, Yang M S (2003) High level of expression of recombinant human granulocyte-macrophage colony stimulating factor in transgenic rice cell suspension culture. Biotechnol Bioengineering 82: 778-783.

Stuiver M H, Custers J H H V (2001) Engineering disease resistance in plants. Nature 411:865-868.

Tesniere C, Pradal M, El-Kereamy A, Torregrosa L, Chatelet P (2004) Involvement of ethylene signaling in a non-climacteric fruit: new elements regarding the regulation of ADH expression in grapevine. J Exp Bot 55: 2235-2240.

Tremousayque D, Manevski A, Bardet C, Lescure N, Lescure B (1999) Plant interstitial telomere motifs participitate in the control of gene expression in root meristems. Plant J 20: 553-561.

Vriezen W H, De Graaf B, Mariani C, Voesenek L A C J (2000) Submergence induces expansin gene expression in flooding-tolerant Rumex palustris and not in flooding-intolerant R. acetosa. Planta 210: 956-963.

Walker J C, Howard E A, Dennis E S, Peacock W J (1987) DNA sequences required for anaerobic expression of the maize alcohol dehydrogenase 1 gene. PNAS 84: 6624-6628.

Wenkel S, Turck F, Singer K, Gissot L, Le Gourrierec J, Samach A, Couplans G (2006) CONSTANS and the CCAAT box binding complex share a functionally important domain and interact to regulate flowering of Arabidopsis. Plant Cell 18: 2971-2984.

Wrobel R L, Yoder J I (2001) Differential RNA expression of α-expansin gene family members in the parasitic angiosperm Triphysaria versicolor (Scrophulariaceae). Gene 266: 85-93.

Zhu Q, Dabi T, Lamb C (1995) TATA box and initiator functions in the accurate transcription of a plant minimal promoter in vitro. Plant Cell 1: 1681-1689.

All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. An isolated nucleic acid molecule comprising a nucleotide sequence selected from the group consisting of:
a) a nucleotide sequence comprising the sequence set forth in SEQ ID NO:1 or a complement thereof; and
b) a nucleotide sequence comprising a fragment of the sequence set forth in SEQ ID NO:1, wherein said sequence initiates transcription in a plant cell; and
c) a nucleotide sequence comprising a sequence having at least 90% sequence identity to the sequence set forth in SEQ ID NO:1, SEQ ID NO:2, or SEQ ID NO:3, wherein said sequence initiates transcription in the plant cell.

2. A DNA construct comprising a nucleotide sequence of claim 1 operably linked to a heterologous nucleotide sequence of interest.

3. A vector comprising the DNA construct of claim 2.

4. A plant cell having stably incorporated into its genome the DNA construct of claim 2.

5. The plant cell of claim 4, wherein said plant cell is from a dicot.

6. The plant cell of claim 5, wherein said dicot is a cherry cultivar.

7. A plant having stably incorporated into its genome the DNA construct of claim 2.

8. The plant of claim 7, wherein said plant is a dicot.

9. The plant of claim 7, wherein said plant is a monocot.

10. A transgenic seed of the plant of claim 7, wherein the seed comprises the DNA construct.

11. The plant of claim 4, wherein the heterologous nucleotide sequence of interest encodes a gene product that confers herbicide, salt, cold, drought, pathogen, or insect resistance or said gene product is of agricultural interest.

12. A method for expressing a nucleotide sequence in a plant, said method comprising introducing into a plant a DNA construct, said DNA construct comprising a promoter and operably linked to said promoter a heterologous nucleotide sequence of interest, wherein said promoter comprises a nucleotide sequence selected from the group consisting of:
a) a nucleotide sequence comprising the sequence set forth in SEQ ID NO:1 or a complement thereof; and
b) a nucleotide sequence comprising a fragment of the sequence set forth in SEQ ID NO:1, wherein said sequence initiates transcription in a plant cell; and
c) a nucleotide sequence comprising a sequence having at least 90% sequence identity to the sequence set forth in SEQ ID NO:1, wherein said sequence initiates transcription in the plant cell.

13. The method of claim 12, wherein said plant is a dicot and wherein said heterologous nucleotide sequence of interest is selectively expressed in a fruit or ovary.

14. The method of claim 12, wherein the heterologous nucleotide sequence encodes a gene product that confers herbicide, salt, cold, drought, pathogen, or insect resistance or said gene product is of agricultural interest.

15. A method for expressing a nucleotide sequence in a plant cell, said method comprising introducing into a plant cell a DNA construct comprising a promoter operably linked to a heterologous nucleotide sequence of interest, wherein said promoter comprises a nucleotide sequence selected from the group consisting of:
a) a nucleotide sequence comprising the sequence set forth in SEQ ID NO:1 or a complement thereof; and
b) a nucleotide sequence comprising a fragment of the sequence set forth in SEQ ID NO:1, wherein said sequence initiates transcription in a plant cell; and
c) a nucleotide sequence comprising a sequence having at least 90% sequence identity to the sequence set forth in SEQ ID NO:1, wherein said sequence initiates transcription in the plant cell.

16. The method of claim 15, wherein said plant cell is from a dicot.

17. The method of claim 15, wherein the heterologous nucleotide sequence encodes a gene product that confers herbicide, salt, cold, drought, pathogen, or insect resistance or said gene product is of agricultural interest.


SEQUENCE LISTING (5 sequences)

SEQ ID NO: 1 - DNA, 989 bp, Prunus cerasus
tcgaaacttattaatttaagagtttaaaagtgtgaaaagagagaggcaaagccaacttggaaggaagacttgtattgccttaattagttaatgctatatagtattcaaggcagaaagaaacatgtgtggggcagtgattaaacataacacatgcactacccaaattgacacacgcaaatgaaataatgggttggttgccacttcctaccatgagccaagcaaatgacattgtgcagagatttggaaaattctgtttgcaaacttacaaagagtgttttagttttacgaaaaaccctaatgcttaaaggttttggtgcgttgtcagggatgcccttttgttcttccttacttgatgagaaggatacttcccaaaagaaaagaaagcatcagaaagtaaaatacttcagaattcaatgctttcattaacaaaattttcagccatgtgcaaggatacccattatctgttaagaccctaccattgattcaacaaatagaagggcattttcaactttcttgggccccaaactaaagattatgccctgtacttaaatttaattaagcacctacaaacagagatcggacacatgttatgagtgcggaatgacaaactgcgagtgtgaataacaactgattggcgccaatcaatgtgatcaaagaaaatctgaatataaaattcaaaataaaaattgtatgaagttgatggctaggaaattgcatgtggagaagtttggcccactggcgtagcggttggtaaccccaaccagaaccagcgcatgtgcacctgatttttctcaatcacttgctgctagggactttctattcaatgaaatgtgtgcaaaactcagtcataagtaatctcaagtttccttataaatagaggccccctcgtgcactctaagcacaaatcaaaacaaacacctcccctgcataaaaaccaaagctcctcctctctcttcctttctcctattgaaaaaatatcaaaaatcatg

SEQ ID NO: 2 - DNA, 606 bp, Prunus cerasus
ggtgcgttgtcagggatgcccttttgttcttccttacttgatgagaaggatacttcccaaaagaaaagaaagcatcagaaagtaaaatacttcagaattcaatgctttcattaacaaaattttcagccatgtgcaaggatacccattatctgttaagaccctaccattgattcaacaaatagaagggcattttcaactttcttgggccccaaactaaagattatgccctgtacttaaatttaattaagcacctacaaacagagatcggacacatgttatgagtgcggaatgacaaactgcgagtgtgaataacaactgattggcgccaatcaatgtgatcaaagaaaatctgaatataaaattcaaaataaaaattgtatgaagttgatggctaggaaattgcatgtggagaagtttggcccactggcgtagcggttggtaaccccaaccagaaccagcgcatgtgcacctgatttttctcaatcacttgctgctagggactttctattcaatgaaatgtgtgcaaaactcagtcataagtaatctcaagtttccttataaatagaggccccctcgtgcactctaagcacaaatcaaaacaaacac

SEQ ID NO: 3 - DNA, 918 bp, Prunus cerasus
tcgaaacttattaatttaagagtttaaaagtgtgaaaagagagaggcaaagccaacttggaaggaagacttgtattgccttaattagttaatgctatatagtattcaaggcagaaagaaacatgtgtggggcagtgattaaacataacacatgcactacccaaattgacacacgcaaatgaaataatgggttggttgccacttcctaccatgagccaagcaaatgacattgtgcagagatttggaaaattctgtttgcaaacttacaaagagtgttttagttttacgaaaaaccctaatgcttaaaggttttggtgcgttgtcagggatgcccttttgttcttccttacttgatgagaaggatacttcccaaaagaaaagaaagcatcagaaagtaaaatacttcagaattcaatgctttcattaacaaaattttcagccatgtgcaaggatacccattatctgttaagaccctaccattgattcaacaaatagaagggcattttcaactttcttgggccccaaactaaagattatgccctgtacttaaatttaattaagcacctacaaacagagatcggacacatgttatgagtgcggaatgacaaactgcgagtgtgaataacaactgattggcgccaatcaatgtgatcaaagaaaatctgaatataaaattcaaaataaaaattgtatgaagttgatggctaggaaattgcatgtggagaagtttggcccactggcgtagcggttggtaaccccaaccagaaccagcgcatgtgcacctgatttttctcaatcacttgctgctagggactttctattcaatgaaatgtgtgcaaaactcagtcataagtaatctcaagtttccttataaatagaggccccctcgtgcactctaagcacaaatcaaaacaaacac

SEQ ID NO: 4 - DNA, 607 bp, Prunus cerasus
agggatgcccttttgttcttccttacttgatgagaaggatacttcccaaaagaaaagaaagcatcagaaagtaaaatacttcagaattcaatgctttcattaacaaaattttcagccatgtgcaaggatacccattatctgttaagaccctaccattgattcaacaaatagaagggcattttcaactttcttgggccccaaactaaagattatgccctgtacttaaatttaattaagcacctacaaacagagatcggacacatgttatgagtgcggaatgacaaactgcgagtgtgaataacaactgattggcgccaatcaatgtgatcaaagaaaatctgaatataaaattcaaaataaaaattgtatgaagttgatggctaggaaattgcatgtggagaagtttggcccactggcgtagcggttggtaaccccaaccagaaccagcgcatgtgcacctgatttttctcaatcacttgctgctagggactttctattcaatgaaatgtgtgcaaaactcagtcataagtaatctcaagtttccttataaatagaggccccctcgtgcactctaagcacaaatcaaaacaaacacctcccctgcata

SEQ ID NO: 5 - DNA, 929 bp, Prunus cerasus
cgaaacttattaatttaagagtttaaaagtgtgaaaagagagaggcaaagccaacttggaaggaagacttgtattgccttaattagttaatgctatatagtattcaaggcagaaagaaacatgtgtggggcagtgattaaacataacacatgcactacccaaattgacacacgcaaatgaaataatgggttggttgccacttcctaccatgagccaagcaaatgacattgtgcagagatttggaaaattctgtttgcaaacttacaaagagtgttttagttttacgaaaaaccctaatgcttaaaggttttggtgcgttgtcagggatgcccttttgttcttccttacttgatgagaaggatacttcccaaaagaaaagaaagcatcagaaagtaaaatacttcagaattcaatgctttcattaacaaaattttcagccatgtgcaaggatacccattatctgttaagaccctaccattgattcaacaaatagaagggcattttcaactttcttgggccccaaactaaagattatgccctgtacttaaatttaattaagcacctacaaacagagatcggacacatgttatgagtgcggaatgacaaactgcgagtgtgaataacaactgattggcgccaatcaatgtgatcaaagaaaatctgaatataaaattcaaaataaaaattgtatgaagttgatggctaggaaattgcatgtggagaagtttggcccactggcgtagcggttggtaaccccaaccagaaccagcgcatgtgcacctgatttttctcaatcacttgctgctagggactttctattcaatgaaatgtgtgcaaaactcagtcataagtaatctcaagtttccttataaatagaggccccctcgtgcactctaagcacaaatcaaaacaaacacctcccctgcata